(19) United States
(12) Patent Application Publication
(10) Pub. No.: US 2010/0272706 A1
(43) Pub. Date: Oct. 28, 2010

(54) ANTIVIRALS

(76) Inventors: Jason Mercer, Zurich (CH); Urs Greber, Maennedorf (CH); Stefan Moese, Zurich (CH); Ari Helenius, Zurich (CH); Lucas Pelkmans, Zurich (CH)

Correspondence Address: WILSON, SONSINI, GOODRICH & ROSATI, 650 PAGE MILL ROAD, PALO ALTO, CA, 94304-1050, US

(21) Appl. No.: 12/143,621

(22) Filed: Jun. 20, 2008

Related U.S. Application Data
(60) Provisional application No. 60/945,740, filed on Jun. 22, 2007.

(51) Int. Cl. A61K 31/553; A61K 31/713; A61K 39/395; C12Q 1/70; A61P 31/12

(52) U.S. Cl. 424/130.1; 514/44 A; 514/211.08; 435/5

(57) ABSTRACT

This invention provides methods for inhibiting or treating infection by viruses, in particular pox viruses by modulating a kinase, in particular by inhibiting a host cell kinase, involved in mediating viral infection. Methods to identify, validate, and classify the cellular proteins required by viruses during infection of host cells in order to select agents which can inhibit viral infection are described herein. Using a systems biology approach the virus/host cell interaction is studied from initial attachment of the incoming virus to the cell surface, to entry, transcription, replication, biosynthesis, and assembly of progeny particles. The method employs a siRNA screening platform and uses gene silencing to map the ‘viral infectome’—a compilation of cellular proteins that the virus needs to establish infection and drive the infectious cycle. Charting the infectome provides information on the viral biology by the identification of host cell proteins involved in viral infection and allows the development of novel anti-viral drugs that prevent the viruses from establishing productive infection in cells.

### CROSS-REFERENCE

This application claims the benefit of U.S. Provisional Application No. 60/945,740, filed Jun. 22, 2007, which is incorporated herein by reference in its entirety.

### BACKGROUND OF THE INVENTION

Antiviral drugs are a class of medication used for the treatment of viral infections. Antiviral drugs are one class of antimicrobials, the larger group of which includes antibiotics, anti-fungals, and anti-parasitic drugs. Unlike antibacterial drugs, which may cover a wide range of pathogens, antiviral agents tend to be narrow in spectrum and have limited efficacy.

The emergence of antivirals is the product of a expanded knowledge of the genetic and molecular function of organisms, allowing biomedical researchers to understand the structure and function of viruses, advances in the techniques for finding new drugs, and the pressure placed on the medical profession to deal with the human immunodeficiency virus (HIV), the cause of the deadly acquired immunodeficiency syndrome (AIDS) epidemic.

With the continuous problem of seasonal human influenza, and the threat of future pandemics, the development of antiviral agents against influenza is a priority (Fauci 2006). Whereas vaccination remains a cornerstone in prophylaxis, antiviral agents constitute an element in the global fight against epidemics and potential pandemics (Pleshka et al. 2006). Anti-viral drugs have advantages over vaccines because their usefulness is unaffected by antigenic changes in the virus, which means that they can be used against emerging strains before vaccines are available. Also, they are effective against established illness. In prophylaxis, they protect against infection, they reduce the spread of virus, and they serve as a useful supplement to immunization. Given these advantages, several United States governmental committees concerned with the risk of influenza pandemics have recommended research to develop novel antiviral agents against this virus.

Currently available antiviral drugs against influenza inhibit either the viral M2-channel (amantidine, rimantidine) or the neuraminidase (NA) (oseltamivir and zanamivir) (Pleshka et al. 2006). While safe and useful for prophylaxis and therapy against human influenza A strains, the former are seldom prescribed. The NA inhibitors are in more common use. They are active against influenza A strains including the avian H5N1, and they also work against influenza B. Oseltamivir is used for prophylaxis and therapy, whereas zanamivir (which is inhaled) is not yet approved for prophylaxis.

The main problem with current antiviral drugs (and drugs that target viral proteins in general) is the emergence of resistance through point mutations in viral genes. For M2-channel blockers, the emergence of full resistance is rapid. In the case of the NA inhibitors, resistant strains do emerge, but fortunately so far they have shown decreased fitness and pathogenicity compared to wild-type. However, the possible generation of resistant strains remains a concern.

Instead of focusing on the virus itself and its proteins as a target, it is advantageous to develop a new generation of anti-viral drugs that interfere with host cell proteins involved in viral infection. By inhibiting the activity of these proteins it will be possible to inhibit the replication and production of progeny virus.

### SUMMARY OF THE INVENTION

The present invention provides methods for identifying host cell proteins which play a role in viral infection. The identification of these host cell target proteins permits the identification of agents that target them for therapeutic interventions for viral infections. Also, provided herein are agents and methods for modulation of these host cell proteins to treat and/or prevent a viral infection. The present invention provides for agents which inhibit or decrease a viral infection in a host cell by modulating a host cell protein. Additionally, the present invention provides for kits that can be used to treat viral infection.

In one aspect, this invention provides a method of treating a poxvirus infection comprising administering to an animal subject in need thereof an effective amount of a kinase modulator. In one embodiment, the animal subject is a human. In one embodiment, said inhibitor of said macropinocytosis pathway is an inhibitor of a kinase selected from the group consisting of PAK1; DYRK3; PTK9; and GPRK2L. In one embodiment, said kinase modulator is a host cell kinase modulator. In one embodiment, the kinase modulator is a dominant negative molecule targeting the kinase, an siRNA, an shRNA an antibody or a small molecule. In one embodiment, the kinase modulator is an siRNA. In one embodiment the kinase modulator is CEP-1347. In one embodiment, said host cell kinase modulator is a host cell kinase inhibitor. In one embodiment, said host cell kinase inhibitor is an inhibitor of a kinase selected from the group consisting of PAK1; DYRK3; PTK9; and GPRK2L. In one embodiment, the poxvirus is a variola virus. In one embodiment, the poxvirus is a vaccinia virus. In one embodiment, the infection is a respiratory infection.

In another aspect, this invention provides a method of treating a virus infection comprising administering to an animal subject in need thereof an effective amount of a modulator of a macropinocytosis pathway. In one embodiment, the animal subject is a human. In one embodiment, said modulator of a macropinocytosis pathway is an inhibitor of said macropinocytosis pathway. In one embodiment, said inhibitor is a kinase inhibitor. In one embodiment, said inhibitor is a host cell kinase inhibitor. In one embodiment, said host cell kinase inhibitor is an inhibitor of a kinase selected from the group consisting of PAK1; DYRK3; PTK9; and GPRK2L. In one embodiment, the inhibitor is CEP-1347. In one embodiment, said virus is a pox virus. In one embodiment, said virus is a variola virus. In one embodiment, said virus is a vaccinia virus.

In another aspect this invention provides a method comprising: contacting a cell with a kinase inhibitor and virus and determining whether the kinase inhibitor inhibits infection of the cell by the virus. In one embodiment kinase inhibitor inhibits a kinase selected from the group consisting of PAK1; DYRK3; PTK9; and GPRK2L. In another embodiment the kinase inhibitor is selected from the group consisting of dominant negative molecule targeting the kinase, an siRNA, an shRNA an antibody or a small molecule. In another embodiment the virus is an influena virus or a pox virus, e.g., vaccinia or variola. In another embodiment the contacting is performed in vitro.

### INCORPORATION BY REFERENCE

All publications, patents and patent applications mentioned in this specification are herein incorporated by reference to the same extent as if each individual publication patent or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts surfing and membrane perturbation during mature virion entry.

FIG. 2 depicts p21-activated kinase-1 (PAK1) is required for MV entry.

FIG. 3 depicts vaccinia MVs utilize macropinocytosis to enter cells.

FIG. 4 depicts vaccinia MVs require PS for internalization.

FIG. 5 depicts activation of PAK1 required for Ad3 but not Ad5 endocytosis and infection.

FIG. 6 depicts a pathway for Ad3 infection using macropinocytosis.

FIG. 7 depicts EGFR activation following MV addition to HeLa cells.

FIG. 8 depicts EGFR inhibitor 324674 (Calbiochem) blocking MV entry, and is by-passed by low-pH fusion.

### DETAILED DESCRIPTION OF THE INVENTION

While multiple embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The methods of the invention include the identification of host cell genes that a virus uses for infection, replication and/or propagation. Also, described herein are methods of identifying agents that target specific host cell proteins, encoded by the identified host cell genes. Further, the present invention includes agents and methods for modulating the identified host cell targets. Such agents and methods are suitable for the treatment of viral infections. Such modulation of host cell targets may include either activation or inhibition of the host cell targets. Accordingly, compounds that modulate, e.g., inhibit, the activity of a non-viral protein, e.g., a host cell protein, e.g., a kinase, are used as antiviral pharmaceutical agents.

In one embodiment the methods of the present invention can be used to develop antivirals to inhibit the infection of an animal subject, such as a human, by any of a plethora of viruses. In one embodiment the methods of the present invention are used to develop antivirals which inhibit the infection of a host by a respiratory virus. Respiratory viruses are most commonly transmitted by airborne droplets or nasal secretions and can lead to a wide spectrum of illness. Respiratory viruses include the respiratory syncytial virus (RSV), influenza viruses, coronaviruses such as SARS, adenoviruses, parainfluenza viruses and rhinoviruses.

### I. Viruses

In one embodiment host cell proteins are identified that a virus, such as a pox virus, an adenovirus or any viruses mentioned herein needs for infection or replication. Adenoviruses most commonly cause respiratory illness; symptoms of respiratory illness caused by adenovirus infection range from the common cold syndrome to pneumonia, croup, and bronchitis. Patients with compromised immune systems are especially susceptible to severe complications of adenovirus infection. Acute respiratory disease (ARD), first recognized among military recruits during World War II, can be caused by adenovirus infections during conditions of crowding and stress. Adenoviruses are medium-sized (90-100 nm), nonenveloped icosohedral viruses containing double-stranded DNA. There are 49 immunologically distinct types (6 subgenera: A through F) that can cause human infections. Adenoviruses are unusually stable to chemical or physical agents and adverse pH conditions, allowing for prolonged survival outside of the body. Some adenoviruses, such as AD2 and Ad5 (species C) use clathrin mediated endocytosis and macropinocytosis for infectious entry. Other adenoviruses, such as Ad3 (species B) use dynamin dependent endocytosis and macropinocytosis for infectious entry.

In one embodiment host cell proteins are identified that a pox virus needs for infection or replication. Pox viruses are generally enveloped. The virus has dimensions of about 200 nm by 300 nm. The DNA is linear and double stranded. The virus Family Poxyiridae includes the genus Orthopoxvirus which includes the species Variola vera, which is responsible for smallpox. The virus comes in two forms, variola major and variola minor. Smallpox typically is transmitted from person to person through inhalation of airborne variola virus, usually from the respiratory system of the infected person. Accordingly, inhibition of these viruses is useful as a defense against bioterrorism. Vaccinia also is an infectious pox virus.

In one embodiment host cell proteins are identified that a respiratory syncytial virus (RSV) needs for infection or replication. RSV is the most common cause of bronchiolitis and pneumonia among infants and children under 1 year of age. Illness begins most frequently with fever, runny nose, cough, and sometimes wheezing. During their first RSV infection, between 25% and 40% of infants and young children have signs or symptoms of bronchiolitis or pneumonia, and 0.5% to 2% require hospitalization. Most children recover from illness in 8 to 15 days. The majority of children hospitalized for RSV infection are under 6 months of age. RSV also causes repeated infections throughout life, usually associated with moderate-to-severe cold-like symptoms; however, severe lower respiratory tract disease may occur at any age, especially among the elderly or among those with compromised cardiac, pulmonary, or immune systems. RSV is a negative-sense, enveloped RNA virus. The virion is variable in shape and size (average diameter of between 120 and 300 nm), is unstable in the environment (surviving only a few hours on environmental surfaces), and is readily inactivated with soap and water and disinfectants.

In one embodiment host cell proteins are identified that a human parainfluenza virus (HPIV) needs for infection or replication. HPIVs are second to respiratory syncytial virus (RSV) as a common cause of lower respiratory tract disease in young children. Similar to RSV, HPIVs can cause repeated infections throughout life, usually manifested by an upper respiratory tract illness (e.g., a cold and/or sore throat). HPIVs can also cause serious lower respiratory tract disease with repeat infection (e.g., pneumonia, bronchitis, and bronchiolitis), especially among the elderly, and among patients with compromised immune systems. Each of the four HPIVs has different clinical and epidemiologic features. The most distinctive clinical feature of HPIV-1 and HPIV-2 is croup (i.e., laryngotracheobronchitis); HPIV-1 is the leading cause of croup in children, whereas HPIV-2 is less frequently detected. Both HPIV-1 and -2 can cause other upper and lower respiratory tract illnesses. HPIV-3 is more often associated with bronchiolitis and pneumonia. HPIV-4 is infrequently detected, possibly because it is less likely to cause severe disease. The incubation period for HPIVs is generally from 1 to 7 days. HPIVs are negative-sense, single-stranded RNA viruses that possess fusion and hemagglutinin-neuraminidase glycoprotein “spikes” on their surface. There are four serotypes types of HPIV (1 through 4) and two subtypes (4a and 4b). The virion varies in size (average diameter between 150 and 300 nm) and shape, is unstable in the environment (surviving a few hours on environmental surfaces), and is readily inactivated with soap and water.

In one embodiment host cell proteins are identified that a coronavirus needs for infection or replication. Coronavirus is a genus of animal virus belonging to the family Coronaviridae. Coronaviruses are enveloped viruses with a positive-sense single-stranded RNA genome and a helical symmetry. The genomic size of coronaviruses ranges from approximately 16 to 31 kilobases, extraordinarily large for an RNA virus. The name “coronavirus” is derived from the Latin corona, meaning crown, as the virus envelope appears under electron microscopy to be crowned by a characteristic ring of small bulbous structures. This morphology is actually formed by the viral spike peplomers, which are proteins that populate the surface of the virus and determine host tropism. Coronaviruses are grouped in the order Nidovirales, named for the Latin nidus, meaning nest, as all viruses in this order produce a 3′ co-terminal nested set of subgenomic mRNA's during infection. Proteins that contribute to the overall structure of all coronaviruses are the spike, envelope, membrane and nucleocapsid. In the specific case of SARS a defined receptor-binding domain on S mediates the attachment of the virus to its cellular receptor, angiotensin-converting enzyme 2.

In one embodiment host cell proteins are identified that a rhinovirus needs for infection or replication. Rhinovirus (from the Greek rhin-, which means “nose”) is a genus of the Picornaviridae family of viruses Rhinoviruses are the most common viral infective agents in humans, and a causative agent of the common cold. There are over 105 serologic virus types that cause cold symptoms, and rhinoviruses are responsible for approximately 50% of all cases. Rhinoviruses have single-stranded positive sense RNA genomes of between 7.2 and 8.5 kb in length. At the 5′ end of the genome is a virus-encoded protein, and like mammalian mRNA, there is a 3′ poly-A tail. Structural proteins are encoded in the 5′ region of the genome and non structural at the end. This is the same for all picornaviruses. The viral particles themselves are not enveloped and are icosahedral in structure.

In one embodiment host cell proteins are identified that an influenza virus needs for infection or replication. Influenza viruses belong to Orthomyxoviridae family of viruses. This family also includes Thogoto viruses and Dhoriviruses. There are several types and subtypes of influenza viruses known, which infect humans and other species. Influenza type A viruses infect people, birds, pigs, horses, seals and other animals, but wild birds are the natural hosts for these viruses. Influenza type A viruses are divided into subtypes and named on the basis of two proteins on the surface of the virus: hemagglutinin (HA) and neuraminidase (NA). For example, an “H7N2 virus” designates an influenza A subtype that has an HA 7 protein and an NA 2 protein. Similarly an “H5N1” virus has an HA 5 protein and an NA 1 protein. There are 16 known HA subtypes and 9 known NA subtypes. Many different combinations of HA and NA proteins are possible. Only some influenza A subtypes (i.e., H1N1, H1N2, and H3N2) are currently in general circulation among people. Other subtypes are found most commonly in other animal species. For example, H7N7 and H3N8 viruses cause illness in horses, and H3N8 also has recently been shown to cause illness in dogs (http://www.cdc.gov/flu/avian/gen-info/flu-viruses.htm).

Antiviral agents which target host cell proteins involved in influenza infection can be used to protect high-risk groups (hospital units, institutes caring for elderly, immuno-suppressed individuals), and on a case by case basis. A potential use for antiviral agents is to limit the spread and severity of the future pandemics whether caused by avian H5N1 or other strains of influenza virus. Avian influenza A viruses of the subtypes H5 and H7, including H5N1, H7N7, and H7N3 viruses, have been associated with high pathogenicity, and human infection with these viruses have ranged from mild (H7N3, H7N7) to severe and fatal disease (H7N7, H5N1). Human illness due to infection with low pathogenicity viruses has been documented, including very mild symptoms (e.g., conjunctivitis) to influenza-like illness. Examples of low pathogenicity viruses that have infected humans include H7N7, H9N2, and H7N2. (http://www.cdc.gov/flu/avian/gen-info/flu-viruses.htm).

Influenza B viruses are usually found in humans but can also infect seals. Unlike influenza A viruses, these viruses are not classified according to subtype. Influenza B viruses can cause morbidity and mortality among humans, but in general are associated with less severe epidemics than influenza A viruses. Although influenza type B viruses can cause human epidemics, they have not caused pandemics. (http://www.cdc.gov/flu/avian/gen-info/flu-viruses.htm).

Influenza type C viruses cause mild illness in humans and do not cause epidemics or pandemics. These viruses can also infect dogs and pigs. These viruses are not classified according to subtype. (http://www.cdc.gov/flu/avian/gen-info/flu-viruses.htm).

Influenza viruses differ from each other in respect to cell surface receptor specificity and cell tropism, however they use common entry pathways. Charting these pathways and identification of host cell proteins involved in virus influenza transmission, entry, replication, biosynthesis, assembly, or exit allows the development of general agents against existing and emerging strains of influenza. The agents may also prove useful against unrelated viruses that use similar pathways. For example, the agents may protect airway epithelial cells against a number of different viruses in addition to influenza viruses.

The methods described herein are useful for development and/or identification of agents for the treatment of infections caused by any virus, including, for example, Abelson leukemia virus, Abelson murine leukemia virus, Abelson's virus, Acute laryngotracheobronchitis virus, Adelaide River virus, Adeno associated virus group, Adenovirus, African horse sickness virus, African swine fever virus, AIDS virus, Aleutian mink disease parvovirus, Alpharetrovirus, Alphavirus, ALV related virus, Amapari virus, Aphthovirus, Aquareovirus, Arbovirus, Arbovirus C, arbovirus group A, arbovirus group B, Arenavirus group, Argentine hemorrhagic fever virus, Argentine hemorrhagic fever virus, Arterivirus, Astrovirus, Ateline herpesvirus group, Aujezky's disease virus, Aura virus, Ausduk disease virus, Australian bat lyssavirus, Aviadenovirus, avian erythroblastosis virus, avian infectious bronchitis virus, avian leukemia virus, avian leukosis virus, avian lymphomatosis virus, avian myeloblastosis virus, avian paramyxovirus, avian pneumoencephalitis virus, avian reticuloendotheliosis virus, avian sarcoma virus, avian type C retrovirus group, Avihepadnavirus, Avipoxvirus, B virus, B19 virus, Babanki virus, baboon herpesvirus, baculovirus, Barmah Forest virus, Bebaru virus, Berrimah virus, Betaretrovirus, Birnavirus, Bittner virus, BK virus, Black Creek Canal virus, bluetongue virus, Bolivian hemorrhagic fever virus, Boma disease virus, border disease of sheep virus, boma virus, bovine alphaherpesvirus 1, bovine alphaherpesvirus 2, bovine coronavirus, bovine ephemeral fever virus, bovine immunodeficiency virus, bovine leukemia virus, bovine leukosis virus, bovine mammillitis virus, bovine papillomavirus, bovine papular stomatitis virus, bovine parvovirus, bovine syncytial virus, bovine type C oncovirus, bovine viral diarrhea virus, Buggy Creek virus, bullet shaped virus group, Bunyamwera virus supergroup, Bunyavirus, Burkitt's lymphoma virus, Bwamba Fever, CA virus, Calicivirus, California encephalitis virus, camelpox virus, canarypox virus, canid herpesvirus, canine coronavirus, canine distemper virus, canine herpesvirus, canine minute virus, canine parvovirus, Cano Delgadito virus, caprine arthritis virus, caprine encephalitis virus, Caprine Herpes Virus, Capripox virus, Cardiovirus, caviid herpesvirus 1, Cercopithecid herpesvirus 1, cercopithecine herpesvirus 1, Cercopithecine herpesvirus 2, Chandipura virus, Changuinola virus, channel catfish virus, Charleville virus, chickenpox virus, Chikungunya virus, chimpanzee herpesvirus, chub reovirus, chum salmon virus, Cocal virus, Coho salmon reovirus, coital exanthema virus, Colorado tick fever virus, Coltivirus, Columbia SK virus, common cold virus, contagious eethyma virus, contagious pustular dermatitis virus, Coronavirus, Corriparta virus, coryza virus, cowpox virus, coxsackie virus, CPV (cytoplasmic polyhedrosis virus), cricket paralysis virus, Crimean-Congo hemorrhagic fever virus, croup associated virus, Cryptovirus, Cypovirus, Cytomegalovirus, cytomegalovirus group, cytoplasmic polyhedrosis virus, deer papillomavirus, deltaretrovirus, dengue virus, Densovirus, Dependovirus, Dhori virus, diploma virus, Drosophila C virus, duck hepatitis B virus, duck hepatitis virus 1, duck hepatitis virus 2, duovirus, Duvenhage virus, Deformed wing virus DWV, eastern equine encephalitis virus, eastern equine encephalomyelitis virus, EB virus, Ebola virus, Ebola-like virus, echo virus, echovirus, echovirus 10, echovirus 28, echovirus 9, ectromelia virus, EEE virus, EIA virus, EIA virus, encephalitis virus, encephalomyocarditis group virus, encephalomyocarditis virus, Enterovirus, enzyme elevating virus, enzyme elevating virus (LDH), epidemic hemorrhagic fever virus, epizootic hemorrhagic disease virus, Epstein-Barr virus, equid alphaherpesvirus 1, equid alphaherpesvirus 4, equid herpesvirus 2, equine abortion virus, equine arteritis virus, equine encephalosis virus, equine infectious anemia virus, equine morbillivirus, equine rhinopneumonitis virus, equine rhinovirus, Eubenangu virus, European elk papillomavirus, European swine fever virus, Everglades virus, Eyach virus, felid herpesvirus 1, feline calicivirus, feline fibrosarcoma virus, feline herpesvirus, feline immunodeficiency virus, feline infectious peritonitis virus, feline leukemia/sarcoma virus, feline leukemia virus, feline panleukopenia virus, feline parvovirus, feline sarcoma virus, feline syncytial virus, Filovirus, Flanders virus, Flavivirus, foot and mouth disease virus, Fort Morgan virus, Four Corners hantavirus, fowl adenovirus 1, fowlpox virus, Friend virus, Gammaretrovirus, GB hepatitis virus, GB virus, German measles virus, Getah virus, gibbon ape leukemia virus, glandular fever virus, goatpox virus, golden shinner virus, Gonometa virus, goose parvovirus, granulosis virus, Gross' virus, ground squirrel hepatitis B virus, group A arbovirus, Guanarito virus, guinea pig cytomegalovirus, guinea pig type C virus, Hantaan virus, Hantavirus, hard clam reovirus, hare fibroma virus, HCMV (human cytomegalovirus), hemadsorption virus 2, hemagglutinating virus of Japan, hemorrhagic fever virus, hendra virus, Henipaviruses, Hepadnavirus, hepatitis A virus, hepatitis B virus group, hepatitis C virus, hepatitis D virus, hepatitis delta virus, hepatitis E virus, hepatitis F virus, hepatitis G virus, hepatitis nonA nonB virus, hepatitis virus, hepatitis virus (nonhuman), hepatoencephalomyelitis reovirus 3, Hepatovirus, heron hepatitis B virus, herpes B virus, herpes simplex virus, herpes simplex virus 1, herpes simplex virus 2, herpesvirus, herpesvirus 7, Herpesvirus ateles, Herpesvirus hominis, Herpesvirus infection, Herpesvirus saimiri, Herpesvirus suis, Herpesvirus varicellae, Highlands J virus, Hirame rhabdovirus, hog cholera virus, human adenovirus 2, human alphaherpesvirus 1, human alphaherpesvirus 2, human alphaherpesvirus 3, human B lymphotropic virus, human betaherpesvirus 5, human coronavirus, human cytomegalovirus group, human foamy virus, human gammaherpesvirus 4, human gammaherpesvirus 6, human hepatitis A virus, human herpesvirus 1 group, human herpesvirus 2 group, human herpesvirus 3 group, human herpesvirus 4 group, human herpesvirus 6, human herpesvirus 8, human immunodeficiency virus, human immunodeficiency virus 1, human immunodeficiency virus 2, human papillomavirus, human T cell leukemia virus, human T cell leukemia virus I, human T cell leukemia virus II, human T cell leukemia virus III, human T cell lymphoma virus I, human T cell lymphoma virus II, human T cell lymphotropic virus type 1, human T cell lymphotropic virus type 2, human T lymphotropic virus I, human T lymphotropic virus II, human T lymphotropic virus III, Ichnovirus, infantile gastroenteritis virus, infectious bovine rhinotracheitis virus, infectious haematopoietic necrosis virus, infectious pancreatic necrosis virus, influenza virus A, influenza virus B, influenza virus C, influenza virus D, influenza virus pr8, insect iridescent virus, insect virus, iridovirus, Japanese B virus, Japanese encephalitis virus, JC virus, Junin virus, Kaposi's sarcoma-associated herpesvirus, Kemerovo virus, Kilham's rat virus, Klamath virus, Kolongo virus, Korean hemorrhagic fever virus, kumba virus, Kysanur forest disease virus, Kyzylagach virus, La Crosse virus, lactic dehydrogenase elevating virus, lactic dehydrogenase virus, Lagos bat virus, Langur virus, lapine parvovirus, Lassa fever virus, Lassa virus, latent rat virus, LCM virus, Leaky virus, Lentivirus, Leporipoxvirus, leukemia virus, leukovirus, lumpy skin disease virus, lymphadenopathy associated virus, Lymphocryptovirus, lymphocytic choriomeningitis virus, lymphoproliferative virus group, Machupo virus, mad itch virus, mammalian type B oncovirus group, mammalian type B retroviruses, mammalian type C retrovirus group, mammalian type D retroviruses, mammary tumor virus, Mapuera virus, Marburg virus, Marburg-like virus, Mason Pfizer monkey virus, Mastadenovirus, Mayaro virus, ME virus, measles virus, Menangle virus, Mengo virus, Mengovirus, Middelburg virus, milkers nodule virus, mink enteritis virus, minute virus of mice, MLV related virus, MM virus, Mokola virus, Molluscipoxvirus, Molluscum contagiosum virus, monkey B virus, monkeypox virus, Mononegavirales, Morbillivirus, Mount Elgon bat virus, mouse cytomegalovirus, mouse encephalomyelitis virus, mouse hepatitis virus, mouse K virus, mouse leukemia virus, mouse mammary tumor virus, mouse minute virus, mouse pneumonia virus, mouse poliomyelitis virus, mouse polyomavirus, mouse sarcoma virus, mousepox virus, Mozambique virus, Mucambo virus, mucosal disease virus, mumps virus, murid betaherpesvirus 1, murid cytomegalovirus 2, murine cytomegalovirus group, murine encephalomyelitis virus, murine hepatitis virus, murine leukemia virus, murine nodule inducing virus, murine polyomavirus, murine sarcoma virus, Muromegalovirus, Murray Valley encephalitis virus, myxoma virus, Myxovirus, Myxovirus multiforme, Myxovirus parotitidis, Nairobi sheep disease virus, Nairovirus, Nanimavirus, Nariva virus, Ndumo virus, Neethling virus, Nelson Bay virus, neurotropic virus, New World Arenavirus, newborn pneumonitis virus, Newcastle disease virus, Nipah virus, noncytopathogenic virus, Norwalk virus, nuclear polyhedrosis virus (NPV), nipple neck virus, O'nyong'nyong virus, Ockelbo virus, oncogenic virus, oncogenic viruslike particle, oncornavirus, Orbivirus, Orf virus, Oropouche virus, Orthohepadnavirus, Orthomyxovirus, Orthopoxvirus, Orthoreovirus, Orungo, ovine papillomavirus, ovine catarrhal fever virus, owl monkey herpesvirus, Palyam virus, Papillomavirus, Papillomavirus sylvilagi, Papovavirus, parainfluenza virus, parainfluenza virus type 1, parainfluenza virus type 2, parainfluenza virus type 3, parainfluenza virus type 4, Paramyxovirus, Parapoxvirus, paravaccinia virus, Parvovirus, Parvovirus B19, parvovirus group, Pestivirus, Phlebovirus, phocine distemper virus, Picodnavirus, Picornavirus, pig cytomegalovirus-pigeonpox virus, Piry virus, Pixuna virus, pneumonia virus of mice, Pneumovirus, poliomyelitis virus, poliovirus, Polydnavirus, polyhedral virus, polyoma virus, Polyomavirus, Polyomavirus bovis, Polyomavirus cercopitheci, Polyomavirus hominis 2, Polyomavirus maccacae 1, Polyomavirus muris 1, Polyomavirus muris 2, Polyomavirus papionis 1, Polyomavirus papionis 2, Polyomavirus sylvilagi, Pongine herpesvirus 1, porcine epidemic diarrhea virus, porcine hemagglutinating encephalomyelitis virus, porcine parvovirus, porcine transmissible gastroenteritis virus, porcine type C virus, pox virus, poxvirus, poxvirus variolae, Prospect Hill virus, Provirus, pseudocowpox virus, pseudorabies virus, psittacinepox virus, quailpox virus, rabbit fibroma virus, rabbit kidney vaculolating virus, rabbit papillomavirus, rabies virus, raccoon parvovirus, raccoonpox virus, Ranikhet virus, rat cytomegalovirus, rat parvovirus, rat virus, Rauscher's virus, recombinant vaccinia virus, recombinant virus, reovirus, reovirus 1, reovirus 2, reovirus 3, reptilian type C virus, respiratory infection virus, respiratory syncytial virus, respiratory virus, reticuloendotheliosis virus, Rhabdovirus, Rhabdovirus carpia, Rhadinovirus, Rhinovirus, Rhizidiovirus, Rift Valley fever virus, Riley's virus, rinderpest virus, RNA tumor virus, Ross River virus, Rotavirus, rougeole virus, Rous sarcoma virus, rubella virus, rubeola virus, Rubivirus, Russian autumn encephalitis virus, SA 11 simian virus, SA2 virus, Sabia virus, Sagiyama virus, Saimirine herpesvirus 1, salivary gland virus, sandfly fever virus group, Sandjimba virus, SARS virus, SDAV (sialodacryoadenitis virus), sealpox virus, Semliki Forest Virus, Seoul virus, sheeppox virus, Shope fibroma virus, Shope papilloma virus, simian foamy virus, simian hepatitis A virus, simian human immunodeficiency virus, simian immunodeficiency virus, simian parainfluenza virus, simian T cell lymphotrophic virus, simian virus, simian virus 40, Simplexvirus, Sin Nombre virus, Sindbis virus, smallpox virus, South American hemorrhagic fever viruses, sparrowpox virus, Spumavirus, squirrel fibroma virus, squirrel monkey retrovirus, SSV 1 virus group, STLV (simian T lymphotropic virus) type I, STLV (simian T lymphotropic virus) type II, STLV (simian T lymphotropic virus) type III, stomatitis papulosa virus, submaxillary virus, suid alphaherpesvirus 1, suid herpesvirus 2, Suipoxvirus, swamp fever virus, swinepox virus, Swiss mouse leukemia virus, TAC virus, Tacaribe complex virus, Tacaribe virus, Tanapox virus, Taterapox virus, Tench reovirus, Theiler's encephalomyelitis virus, Theiler's virus, Thogoto virus, Thottapalayam virus, Tick borne encephalitis virus, Tioman virus, Togavirus, Torovirus, tumor virus, Tupaia virus, turkey rhinotracheitis virus, turkeypox virus, type C retroviruses, type D oncovirus, type D retrovirus group, ulcerative disease rhabdovirus, Una virus, Uukuniemi virus group, vaccinia virus, vacuolating virus, varicella zoster virus, Varicellovirus, Varicola virus, variola major virus, variola virus, Vasin Gishu disease virus, VEE virus, Venezuelan equine encephalitis virus, Venezuelan equine encephalomyelitis virus, Venezuelan hemorrhagic fever virus, vesicular stomatitis virus, Vesiculovirus, Vilyuisk virus, viper retrovirus, viral haemorrhagic septicemia virus, Visna Maedi virus, Visna virus, volepox virus, VSV (vesicular stomatitis virus), Wallal virus, Warrego virus, wart virus, WEE virus, West Nile virus, western equine encephalitis virus, western equine encephalomyelitis virus, Whataroa virus, Winter Vomiting Virus, woodchuck hepatitis B virus, woolly monkey sarcoma virus, wound tumor virus, WRSV virus, Yaba monkey tumor virus, Yaba virus, Yatapoxvirus, yellow fever virus, and the Yug Bogdanovac virus. In one embodiment an infectome will be produced for each virus that includes an inventory of the host cellular genes involved in virus infection during a specific phase of viral infection, such cellular entry or the replication cycle.

### II. Viral Infection Pathways

The host cell targets disclosed herein preferably play a role in the viral replication and/or infection pathways. Targeting of such host cell targets modulates the replication and/or infection pathways of the viruses. In preferred embodiments the identified host cell targets are directly or indirectly modulated with suitable agents. Such suitable agents may include small molecule therapeutics, protein therapeutics, or nucleic acid therapeutics. The modulation of such host cell targets can also be performed by targeting entities in the upstream or downstream signaling pathways of the host cell targets.

Like other viruses, the replication of influenza virus involves six phases; transmission, entry, replication, biosynthesis, assembly, and exit. Entry occurs by endocytosis, replication and vRNP assembly takes place in the nucleus, and the virus buds from the plasma membrane. In the infected patient, the virus targets airway epithelial cells. Preferably, in the methods described herein, at least one host cell target involved in such pathways is modulated.

For some viruses a great deal of progress has been made in the elucidation of the steps involved during infection of host cells. For example, experiments initiated in the early 1980s showed that influenza virus follows a stepwise, endocytic entry program with elements shared with other viruses such as alpha- and rhabdoviruses (Marsh and Helenius 1989; Whittaker 2006). The steps include: 1) Initial attachment to sialic acid containing glycoconjugates receptors on the cell surface; 2) signaling induced by the virus particle; 3) endocytosis by clathrin-dependent and clathrin-independent cellular mechanism; 4) acid-induced, hemaglutinin (HA)-mediated penetration from late endosomes; 5) acid-activated, M2 and matrix protein (M1) dependent uncoating of the capsid; and, 6) intra-cytosolic transport and nuclear import of vRNPs. These steps depend on assistance from the host cell in the form of sorting receptors, vesicle formation machinery, kinase-mediated regulation, organelle acidification, and, most likely, activities of the cytoskeleton.

Influenza attachment to the cells surface occurs via binding of the HA1 subunit to cell surface glycoproteins and glycolipids that carry oligosaccharide moieties with terminal sialic acid residues (Skehel and Wiley 2000). The linkage by which the sialic acid is connected to the next saccharide contributes to species specificity. Avian strains including H5N1 prefer an a-(2,3)-link and human strains a-(2,6)-link (Matrosovich 2006). In epithelial cells, binding occurs preferentially to microvilli on the apical surface, and endocytosis occurs at base of these extensions (Matlin 1982). Whether receptor binding induces signals that prepare the cell for the invasion is not yet known, but it is likely because activation of protein kinase C and synthesis of phosphatidylinositol-3-phosphate (PI3P) are required for efficient entry (Sieczkarski et al. 2003; Whittaker 2006).

Endocytic internalization occurs within a few minutes after binding (Matlin 1982; Yoshimura and Ohnishi 1984). In tissue culture cells influenza virus makes use of three different types of cellular processes; 1) preexisting clathrin coated pits, 2) virus-induced clathrin coated pits, and 3) endocytosis in vesicles without visible coat (Matlin 1982; Sieczkarski and Whittaker 2002; Rust et al. 2004) see also results). Video microscopy using fluorescent viruses showed, the virus particles undergoing actin-mediated rapid motion in the cell periphery followed by minus end-directed, microtubule-mediated transport to the perinuclear area of the cell. Live cell imaging indicated, that the virus particles first entered a subpopulation of mobile, peripheral early endosomes that carry them deeper into the cytoplasm before penetration takes place (Lakadamyali et al. 2003; Rust et al. 2004). The endocytic process is regulated by protein and lipid kinases, the proteasome, as well as by Rabs and ubiquitin-dependent sorting factors (Khor et al. 2003; Whittaker 2006).

The membrane penetration step is mediated by low pH-mediated activation of the trimeric, metastable HA, and the conversion of this Type I viral fusion protein to a membrane fusion competent conformation (Maeda et al. 1981; White et al. 1982). This occurs about 16 min after internalization, and the pH threshold varies between strains in the 5.0-5.6 range. The target membrane is the limiting membrane of intermediate or late endosomes. The mechanism of fusion has been extensively studied (Kielian and Rey 2006). Further it was observed that fusion itself does not seem to require any host cell components except a lipid bilayer membrane and a functional acidification system (Maeda et al. 1981; White et al. 1982). The penetration step is inhibited by agents such as lysosomotropic weak bases, carboxylic ionophores, and proton pump inhibitors (Matlin 1982; Whittaker 2006).

To allow nuclear import of the incoming vRNPs, the capsid has to be disassembled. This step involves acidification of the viral interior through the amantadine-sensitive M2-channels causes dissociation of M1 from the vRNPs (Bukrinskaya et al. 1982; Martin and Helenius 1991; Pinto et al. 1992). Transport of the individual vRNPs to the nuclear pore complexes and transfer into the nucleus depends on cellular nuclear transport receptors (O'Neill et al. 1995; Cros et al. 2005). Replication of the viral RNAs (synthesis of positive and negative strands), and transcription occurs in complexes tightly associated with the chromatin in the nucleus. It is evident that, although many of the steps are catalyzed by the viral polymerase, cellular factors are involved including RNA polymerase activating factors, a chaperone HSP90, hCLE, and a human splicing factor UAP56. Viral gene expression is subject to complex cellular control at the transcriptional level, a control system dependent on cellular kinases (Whittaker 2006).

The final assembly of an influenza particle occurs during a budding process at the plasma membrane. In epithelial cells, budding occurs at the apical membrane domain only (Rodriguez-Boulan 1983). First, the progeny vRNPs are transported within the nucleoplasm to the nuclear envelope, then from the nucleus to the cytoplasm, and finally they accumulate in the cell periphery. Exit from the nucleus is dependent on viral protein NEP and M1, and a variety of cellular proteins including CRM1 (a nuclear export receptor), caspases, and possibly some nuclear protein chaperones. Phosphorylation plays a role in nuclear export by regulating M1 and NEP synthesis, and also through the MAPK/ERK system (Bui et al. 1996; Ludwig 2006).

The three membrane proteins of the virus are synthesized, folded and assembled into oligomers in the ER (Doms et al. 1993). They pass through the Golgi complex; undergo maturation through modification of their carbohydrate moieties and proteolytic cleavage. After reaching the plasma membrane they associate with M1 and the vRNPs in a budding process that results in the inclusion of all eight vRNPs and exclusion of most host cell components except lipids.

Influenza infection is associated with activation of several signaling cascades including the MAPK pathway (ERK, JNK, p38 and BMK-1/ERK5), the IkB/NF-kB signaling module, the Raf/MEK/ERK cascade, and programmed cell death (Ludwig 2006). These result in a variety of effects that limit the progress of infection such as transcriptional activation of IFNb, apoptotic cell death, and a block in virus escape of from late endosomes (Ludwig 2006).

Most previous studies on virus-cell interactions were performed in tissue culture using tissue culture- or egg-adapted virus strains. The viruses in these examples were adapted in such as manner that changes were induced that affected receptor binding and tropism (Matrosovich 2006). Infection with wild-type pathogenic strains is provides a more natural picture of viral interaction with host proteins. It is known that in the human airways influenza A and B primarily infect non ciliated epithelial cells in the upper respiratory track carrying NeuSAc a-(2,6)-Gal, whereas avian strains infect ciliated epithelial cell with a-(2,3)-linked sialic acids deeper in the airways (Matrosovich et al. 2004a).

III. Viral Entry into Cells VIa Macropinocytosis

One aspect of the invention is antiviral therapy targeted at proteins involved in the macropinocytosis viral entry pathway. Preferably the target proteins are host cell proteins. Preferred targets are kinases and proteins in the kinase pathways. Preferred targets include PAK1; DYRK3; PTK9; GPRK2L; Cdc42; and/or Rac1. Preferably, the macropinocytosis pathway is targeted for the treatment of poxvirus infections. A preferred poxvirus is the variola virus, the causative agent of smallpox.

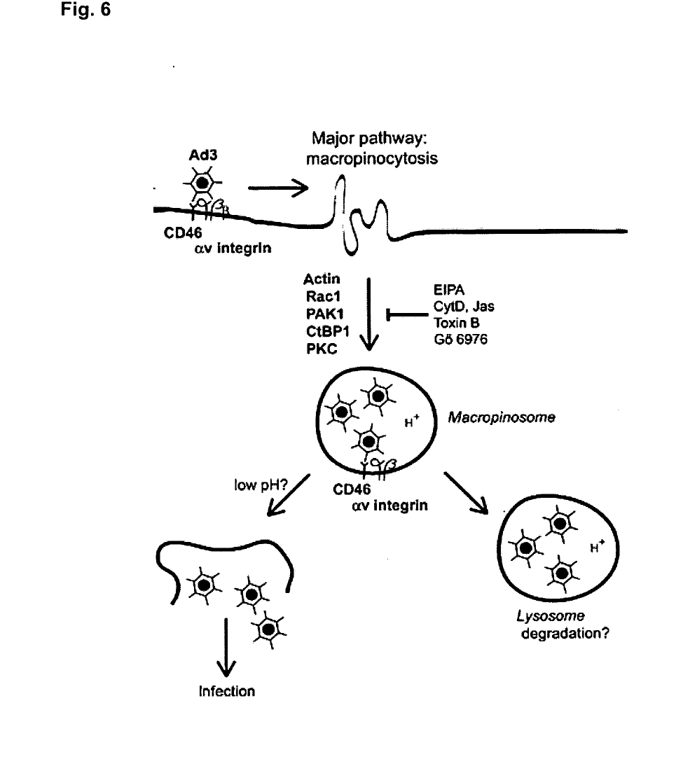

Not intending to be limited to a single mechanism of action, it has been observed that pox viruses, e.g., vaccinia virus, uses macropinocytosis and apoptotic mimicry to enter host cells FIG. 6. Macropinocytosis is a process by which large volumes of fluid are enclosed and internalized. The pathway involves plasma membrane reorganization, formation of endocytic vesicles, and the closure of lamellipodia at the sites of membrane ruffling to form macropinosomes (Lanzavecchia, A (1996) Curr Opin Immunol 8 348-354; Sieczkarski and Whittaker (2002) J Gen Virol 83: 1535-1545). Rho GTPases (West et al. (2000) Curr Biol 10:839-848), ARF6 (Radhakrishna et al. (1996) J Cell Biol 134:935-947), and type 1 phosphatidylinositol-3 kinases (PI3-Ks) (Hooshmand-Rad et al. (1997) Exp Cell Res 234:434-441) are involved in macropinocytosis. In addition, two Rab GTPases, Rab5 and Rab34/Rah, are implicated in the formation of macropinosomes (Li et al. (1997) J Biol Chem 272:10337-10340; Sun et al. (2003) J Biol Chem 278:4063-4071). Rab34/Rah can colocalize with actin to membrane ruffles and nascent macropinosomes, and its overexpression can promote macropinocytosis (Sun et al. (2003) J Biol Chem 278:4063-4071). Rab5 can colocalize to macropinosomes with Rab34/Rah (Sun et al. (2003) J Biol Chem 278:4063-4071).

The inventors analyzed the mechanism used by vaccinia virus, the prototype poxvirus, to enter its host cells. It was observed that the viruses bound to filopodia of tissue culture cells, they moved along these towards the cell body, and they induced the extrusion of large, transient membrane blebs containing actin and actin-associated proteins. As the blebs retracted, the viruses were internalized in endocytic vacuoles. The inhibitors such as blebbistatin, EIPA, or dominant negative constructs of PAK1 that inhibited bleb-formation also blocked infection. The inhibition profile revealed that the MVs induced macropinocytosis, an endocytic process involved in the elimination of cell remnants after apoptosis. Since the determinant for internalization of apoptotic bodies by macropinocytosis is exposure of phosphatidylserine (PS), and since the membrane of MVs is unusually rich in this phospholipid, viruses with different lipid compositions were prepared. The presence of PS played a role in bleb formation, macropinocytosis, and infectivity. The results indicate that the vaccinia virus uses virus-induced macropinocytosis and apoptotic mimicry.

In another embodiment, the macropinocytosis pathway is targeted for the treatment of adenoviruses, preferably species B human adenovirus serotype 3 (Ad3) which is associated with epidemic conjunctivitis, exacerbations of asthmatic conditions, mobidity and mortality. Preferably the therapeutics against adenoviruses target actin, protein kinase C, sodium-proton exchanger, Rac1, PAK1, and the C-terminal adenoviral E1A binding protein-1 (CtBP1).

Not intending to be limited to a single mechanism of action, it has been observed that that Ad3 uses dynamin-independent macropinocytosis for entry into epithelial and hematopoietic cells. Infectious Ad3 macropinocytosis is sensitive to inhibitors targeting actin, protein kinase C, sodium-proton exchanger, and Rac1 but not Cdc42. It requires viral activation of p21-activated kinase 1 (PAK1), and the C-terminal adenoviral E1A binding protein-1 (CtBP1), a bifunctional protein involved in membrane traffic and transcriptional repression, including innate immune responses. CtBP1 is phosphorylated by PAK1, and recruited to the plasma membrane and macropinosomes coincident with transcriptional derepression. Together, Ad3 subverts an innate endocytic immune pathway designed for antigen presentation, which broadens viral host range at the cost of transcriptional anti-viral host gene activation.

IV. Methods and Apparatus for Identification of Host Cell Proteins that Play a Role in Viral Infection.

Cell invasion and productive infection by viruses involves a step-wise program where a few of the events are mediated by viral proteins and enzymes, but the rest depends on cellular functions. To obtain a complete inventory of the cellular proteins involved, embodiments utilizing a systems biology approach are quite useful. Embodiments involving the systematic identification of essential genes involved in influenza infection in tissue culture cells provide an informative avenue of discovery. Systems biology approaches involving genome-wide libraries of siRNAs, and high-throughput instrument platforms can quickly and efficiently identify host cell proteins involved in viral infection from a plethora of candidate proteins.

In one embodiment systematic identification of host cell proteins is performed with the use of an automated high-throughput siRNA screening technology combined with the genomic data base information. Wherein, the genomic database may be derived from any species for whose genomic sequence is known, including the human, the mouse, or an avian species. In some embodiments a screening platform with advanced robotics and screening technology with such as the RNAi Image-based Screening Center' (RISC), may be used. The siRNA screening can be practiced using any suitable host cells or cell lines, including mouse or human host cells, such as airway epithelial cells, or host cell lines, such as HeLa MZ cells, HeLa Kyoto cells, or A549 cells. Other suitable cell lines include a bronchial cell line called 16HBE, a tracheal cell line called THE, as well as commercially available human airway epithelial cell cultures that form well-differentiated pseudostratified mucociliary epithelia in culture (HBEpC, purchased from Promocell, Heidelberg Germany) at an air-liquid interphase (in so called ALI cultures). It is known that such cells can be used as models for influenza infection (Matrosovich et al. 2004). In some embodiments a stable host cell line transformed to express a relevant or required viral entry receptor (for example. CD4 and CXCR4 for HIV-1) may be produced. The host cells may be screened using a genomic library of siRNAs previously validated for functional efficacy. In some embodiments, the genomic library of siRNAs may be obtained from a commercial source such as Qiagen.

In one embodiment HeLa cells are used as the host cells. HeLa cells allow efficient silencing by siRNA transfection. Embodiments involving the testing of influenza viruses demonstrate that single influenza viruses bind to the plasma membrane both in coated and uncoated pits. At 10 min, viruses are present in coated and uncoated small vesicles, and after 30 min many were detected in larger vesicles with an appearance consistent with endosomes. The morphology of virus entry thus resembles that observed in MDCK cells except internalization is slower. Further the trajectories of influenza viruses into and out of endosomal structures were traced using Hela cells which express Rab5-GFP, which marks the early endosomes green, and Rab7-RFP which makes late endosomes red. Further, HeLa cells are to be used to study early stages of infection, transcription, and viral protein synthesis or to screen for defects in some of the later steps such as vRNP export from the nucleus.

In another embodiment A549 cells are used as the host cells. A549 cells are especially useful in embodiments involving respiratory virus infection studies, such as the influenza virus. A549 cells are an epithelial cell line of bronchial origin that has been widely used for influenza infection studies (Ehrhardt et al. 2006). The A549 cells provide a system more similar to the host cells infected in situ during influenza disease. Additionally A549 cells offer possibilities to analyze the whole replication cycle including progeny virus release and secondary infection. Unlike MDCK cells often used in influenza studies and assays, the A549 cells are of human origin and they are easily transfected by siRNAs (Graeser 2004). In a further embodiment, two influenza viruses are tested to analyze the spread of virus and secondary infection in A549 cultures in automated high-throughput formats: 1) an avian H7N7 virus the HA of which is activated by secretase cleavage in most cell lines (Wurzer et al. 2003); and 2) a human influenza strain such as the X31/Aichi/68 and a trypsin overlay formulation that is compatible with use in 96, 384, 768, 1152, 1440, 1536, 3072 well plates, or other multiwell plate formats.

It is further contemplated that embodiments of the invention can be practiced using an automated screening platform. Wherein, the screening platform may comprise a liquid handling robot, such as a Tecan and two automated microscopes, such as the CellWorx, from Applied Precision Instruments. It is anticipated that the automated screening platform can be used to perform high-throughput experimental procedures. Further, computational and experimental efforts may be combined in parallel, to optimally adapt the siRNA assays and to set-up software for fully automated data tracking, image analysis, quantification, and statistical analysis.

In some large scale embodiments screens with siRNAs covering the entire genome of the host cell line are performed. In other embodiments screens with siRNAs covering a subset of the genome (such as at least, 600, 100, 2000, 3000, 4000, 5000, 6000, 7000, 8000, 9000, 10000, 15000, 20000, 25000, or 30000 genes) of the host cell line are performed. For example, in one possible embodiment a screen with siRNAs covering at least 7,000 genes of the human genome is performed. The RISC platform allows a 7,000-gene screen to be completed in 2-4 weeks with two different cell lines for each virus strain studied. Custom-made MatLab plug-ins are then be used to thoroughly analyze and control the quality of the datasets. MatLab plug-ins allow automatic quantification of data in the images generated, and may contain quality control algorithms that automatically discard poor quality images and determine the robustness and reproducibility of the data analysis. Once analysis is completed the results allow the identification of the host proteins involved in viral entry. The viral infectome library builds on bioinformatics tools originally generated for the analysis of cDNA microarrays, but extensively modified for use with RNAi datasets. Robust statistics of large datasets insures that the most weight is given to highly significant phenotypes. Particular phenotypes are weighted by using at least three siRNAs for each gene tested and requiring that 2 out of 3 siRNAs against a gene show similar effects.

Some embodiments may employ an image-based assay that is more sensitive than plate-readers, and therefore yields additional information about the cell biology behind viral infection. In these embodiments high sensitivity is desired since on average only 10-20% of cells may be infected in the unperturbed control. A low ‘base line’ is related to more efficient siRNA silencing, and to differentiate between an increase and decrease in infection. This determination provides optimal information about infection pathways.

In some embodiments involving large format siRNA screens (e.g. large gene sets that cover a subset of, or the entire host cell genome) an automated liquid handling robot, such as a Tecan, which can handle 96, 384, 768, 1152, 1440, 1536, 3072 well plates, or other multiwell plates is used. Algorithms that automatically move the data generated (9 images per well; 1,430,784 images per screen, corresponding to app. 3.8 TB) to a NAS server are be used. In further embodiments a high buffer capacity, such as 1, 2, 3, 4, 5, 6, 7, 8, 9, or 10 TB, guarantees that temporary network failures will not slowdown the analytic process. In further embodiments algorithms that continuously search these large sets of images for non-analyzed images automatically place images into the analysis queue. In some of these embodiments MatLab image analysis plug-ins are used. Further, the ‘raw’ data from the screens may be subjected to bioinformatics evaluation, to screen out false positives, which allows reconstruction of the cellular systems involved in the complex process. This will allows the definition of key target host cell proteins of the molecular machinery specific for each entry route and other infection-related processes. In some embodiments the criteria used includes strong RNAi phenotypes and wide cell-type dependency.

The methods described above have been performed with siRNAs. However other suitable molecular entities may be used such as, organic or inorganic compounds, proteins such as antibodies, or nucleic acid entities such as anti-sense RNA.

The nucleic acid therapeutics of this invention can be natural nucleic acids, modified nucleic acids or analogs of nucleic acids. Nucleic acid analogs, include, for example, peptide nucleic acids (PNA), locked nucleic acids (LNA), threose nucleic acids (TNA), expanded base DNA (xDNA or yDNA). Similarly, phosphorothioate or phosphonate backbone-modified nucleic acids are also encompassed.

### V. Kinases

In some embodiments the host cell proteins identified that modulate viral infection are kinases. In some further embodiments the host cell proteins are PAK1, Cdc42, Rac1, DYRK3, PTK9, and GPRK2L. Several hundred human kinases are known, which map to several different families and are known to play roles in a variety of disease states (Manning et al. 2002).

Inhibitors of kinases include, for example, dominant negative molecules, siRNAs, shRNAs, antibodies and small molecules. Dominant negative molecules include molecules that interfere with the in vitro or in vivo function of a protein by, for example, blocking intramolecular or intermolecular protein-protein interaction interfaces. Dominant negative molecules include, for example, fragments of a protein target (including mutant fragments) and non-functional mutants of a target protein. Antibodies include, for example, complete immunoglobulins, single chain antibodies and specific binding portion of an immunoglobulin. Small molecules include, for example, organic or inorganic non-polymeric molecules having masses up to about 5000 Da, up to about 2000 Da, or up to about 1000 Da.

A. PAK1

There are many insights into the molecular mechanism of PAK1 function (reviewed in Parrini, M C et al. (2005) Biochem Soc Trans 33: 646-648). PAK1 can exist as an auto-inhibited homodimer (Lei, M et al. (2000) Cell 102, 387-397). The N-terminal regulatory domain of one PAK1 molecule can bind to and inhibit the catalytic domain in the C-terminal terminus of another PAK1 molecule. PAK1 can be activated by binding GTP-bound forms of Cdc42 and Rac1. Binding to these molecules can alter the folding of the regulatory domain, leading to dissociation of a PAK1 homodimer (Lei, M et al. (2000) supra; Parrini, M C et al. (2002) Mol. Cell. 9, 73-83). PAK1 can also bind and be activated by the GTPases Rac2, Rac3, TC10, CHP, and Wrch-1 (reviewed in Zhao and Manser (2005) Biochem J. 386, 201-214). Binding and activation by these GTPases can be mediated by residues in the N-terminal regulatory domain or PBD (p21-binding domain). Cdc42 and Rac can bind minimally to the Cdc42 and Rac interactive binding domain (CRIB) (Burbelo et al. (1995) J Biol. Chem. 8, 29071-29074) of PAK1 (amino acids 75-90), and sequences in the flanking kinase inhibitory domain (KI) can contribute to binding affinity (Knaus and Bokoch (1998) Int J Biochem Cell Biol. 30, 857-862; Sells, Mass. and Chernoff, J (1997) Trends Cell Biol. 7, 162-167; Lei, M et al. (2000) supra). A short lysine-rich segment (PAK1 amino acids 66-68) N-terminal of the CRIB domain can mediate Rac GTPase binding (Knaus, U G and Bokoch G M (1998) supra).

The KI domain can inhibit the catalytic domain with a Ki of ˜90 nM (Zhao et al. (1998) Mol. Cell. Biol. 18:2153-2163). The PAK1 KI domain residue Leu-107, as well as other amino acids of the KI domain, can contribute to this inhibitory interface (Lei et al. (2000) supra). The KI region of PAK1 can stabilize two structural components of the active site (helix C and the activation loop). A lysine from the KI segment can block the active site by forming salt bridges with two aspartate residues that play a role in catalysis. This KI polypeptide can block PAK activation (Zhao et al. (1998) supra). The binding constants for binding of peptides including the PAK1 PBD to Cdc42 to have been reported to be in the range of 10-50 nM (Thompson et al. (1998) Biochemistry 37:7885-7891).

The N-terminal regulatory domain of PAK1 also contains two conserved PXXP SH3 (Src homology 3) binding motifs and a conserved SH3 binding site that can bind the PAK-interacting exchange factor (PIX) (Manser et al. (1998) Mol. Cell 1:183-192). The first conserved SH3 binding site can bind the adaptor protein Nck (Bokoch et al. (1996) J. Biol. Chem. 271:25746-25749) and the second can bind Grb2 (Puto et al. (2003) J. Biol. Chem. 278: 9388-9393).

It has been suggested that GTPase binding can cause a change in the conformation of the KI domain that disrupts its interaction with the catalytic domain, allowing autophosphorylation that can contribute to kinase activity (Lei, M et al. (2000) supra). Autophosphorylation can switch PAK1 to an active state. Autophosphorylation of Thr-423 of PAK1 in the catalytic domain in the activation loop can maintain relief from auto-inhibition and for catalytic function towards exogenous substrates (Yu et al. (1998) Biochem. J. 334:121-131; Gatti et al. (1999) J. Biol. Chem. 274:8022-8028; Zenke et al. (1999) J. Biol. Chem. 274:32565-32573); PAK1 may be modified by PDK1 (3-phosphoinositide-dependent kinase 1) (King et al. (2000) J. Biol. Chem. 275:41201-41209). Autophosphorylation of αPAK at Ser-144 (a conserved residue in the KI domain) can contribute to kinase activation (Chong et al. (2001) J. Biol. Chem. 276:17347-17353), while autophosphorylation sites Ser-198/203 of PAK1 can down-regulate the PIX-PAK interaction.

PAK1 can be activated independently of Rae and Cdc42 GTPases. Limited protease-mediated digestion can stimulate PAK kinase autophosphorylation and activity (Brenner et al. (1995) J. Biol. Chem. 270:21121-21128; Roig et al. (1998) Vitam. Horm. 62, 167-198). Membrane recruitment of PAK1 via SH3-containing Nck and Grb2 adaptor proteins can stimulate kinase activity (Lu et al. (1997) Curr. Biol. 7 85-94; Daniels et al. (1998) EMBO J. 274:6047-6050). This activation might involve phosphorylation at the critical Thr-423 residue by PDK1 (King et al. (2000) J. Biol. Chem. 275:41201-41209) or interaction with lipids such as sphingosine, which can activate the kinase in a GTPase-independent manner (Bokoch et al. (1998) J. Biol. Chem. 273:8137-8144). GIT1 (G-protein-coupled receptor kinase-interacting target 1), which can associate indirectly with PAK via PIX, can also activate PAKs through a mechanism that does not require Rho GTPases (Loo et al. (2004) Mol. Cell. Biol. 24:3849-3859).

PAK1 can form a complex with the focal adhesion-associated protein PIX (also referred to as Cool). Multiple PIX proteins, derived from two different genes (αPIX and βPIX), can bind PAK via their SH3 domains (Manser et al. (1998) Mol. Cell. 1:183-192; Bagrodia et al. (1999) J. Biol. Chem. 274:22393-22400). A role for the PIX-PAK complex in the Cdc42-mediated direction sensing of chemotactic leucocytes has been suggested from analysis of cells lacking αPIX (Li et al. (2003) Cell 114:215-227). PIX association with GIT1 (also known as PKL/CAT1 (Turner et al. (1999) J. Cell Biol. 145:851-863; Bagrodia et al. (1999) supra) can target focal adhesions by binding paxillin (Turner et al. (1999) supra). Overexpression of GIT1 can result in disassembly of focal adhesions and a loss of paxillin (Loo et al. (2004) Mol. Cell. Biol. 24:3849-3859). Thus, GIT1 and PIX can both localize and activate PAK at focal adhesions, at the leading edge of motile cells, and to cell-cell junctions (Zegers et al. (2003) EMBO J. 22:4155-4165; Zhao et al. (2000) Mol. Cell. Biol. 20:6354-6363; Manabe et al. (2002) J. Cell Sci. 115:1497-1510).

Two related human protein phosphatases can dephosphorylate PAK1, including at Thr-423 (Koh et al. (2002) Curr. Biol. 12:317-321). These phosphatases are POPX1 (partner of PIX 1) and POPX2, which can bind to different forms of PIX and form multimeric complexes that contain PAK. The effects of active PAK1 in a cell can be antagonized by overexpression of either of these phosphatases (Manabe et al. (2002) supra).

Other protein kinases might down-regulate PAK function. Akt can phosphorylate PAK1 at Ser-21, and this modification can decrease binding of Nck to the PAK1 N-terminus while increasing kinase activity (Zhao et al. (2000) supra; Tang et al. (2000) J. Biol. Chem. 275:9106-9109).

PAK1 is involved in regulating macropinocytosis. An activated PAK1 mutant (T423E) can trigger the dissolution of stress fibers and focal adhesion complexes, the formation of lamellipodia (Sells et al. (1997) Curr Biol. 7: 202-210; Manser et al. (1997) Mol Cell Biol 17:1129-1143), and reorganization of the actin cytoskeleton. Kinase activity and protein-protein interactions involving PAK1 can affect the actin cytoskeleton (Sells et al. (1997) Curr Biol. 7: 202-210; Turner et al. (1999) J Cell Biol. 145: 851-863).

Inhibitors of PAK1 that can be used in the methods and compositions of the present invention include, for example, a dominant negative version of PAK1 containing the PAK1 residues 1-74 which can modulate endothelial cell migration (MSNNGLDIQD KPPAPPMRNT STMIGAGSKD AGTLNHGSKP LPPNPEEKKK KDRFYRSILP GDKTNKKKEK ERPE; (SEQ ID NO:1) (Kiosses et al. (1999) J. Cell Biol. 147:831-843); 13 amino acids from the first proline-rich domain of PAK1 (KPPAPPMRNTSTM; (SEQ ID NO: 2)); these residues fused to the polybasic sequence of HIV tat protein (YGRKKRRQRRRGKPPAPPMRNTSTM; (SEQ ID NO: 3)) (Kiosses et al. (2002) Circ. Res. 90:697-702); a fragment of PAK1 spanning amino acids 83-149, HTIHVGFDAV TGEFTGMPEQ WARLLQTSNI TKSEQKKNPQ AVLDVLEFYN SKKTSNSQKY MSFTDKS (SEQ ID NO: 4), which contains the PAK1 autoinhibitory domain and can block macropinocytosis (Dharmawardhane et al. (2000) Mol. Biol. Cell 11:3341-3352; Barradeau et al. U.S. Pat. No. 7,364,887); peptides of dynein light chain-1/protein inhibitor of nitric oxide synthase (DLC1/PIN) that can affect binding with PAK1 (Kumar et al. U.S. Pat. No. 7,067,633); dominant-negative PAK1 (PAK1 (K299R)) (Naor U.S. Patent Application Publication No. 20050090474).

Indirect inhibitors of Pak1 that can be used in the methods and compositions of the present invention include, for example, the histone deacetylase inhibitor FK228, which can reduce PAK1 kinase activity (Hirokawa et al. (2005) Can. Biol. Ther. 4:956-960); the tyrosine-kinase inhibitors PP1 and AG879, which can reduce PAK1 activation by inhibiting a Src family kinase and ETK, respectively (He et al. (2004) Can. Biol. Ther. 3:96-101; He et al. U.S. Patent Application Publication No. 20030153009); and the combination of PP1 and a water-soluble derivative of AG 879, GL-2003, which also reduces PAK1 activity (Hirokawa et al. (2006) Cancer Letters 245:242-251).

Another inhibitor of PAK1 that can be used in the methods and compositions of the present invention includes CEP-1347, a direct inhibitor of PAK1 in vitro and in vivo (Nheu, T V et al. (2002) Cancer J. 8, 328-336).

Additional inhibitors of PAK1 that can be used in the methods and compositions of the present invention include those disclosed in Van Eyk et al. U.S. Pat. No. 6,248,549.

Additional inhibitors of PAK1 that can be used in the methods and compositions of the present invention include siRNAs against PAK1: siPAK1-0 AGAGCTGCTACAGCATCAA (SEQ ID NO: 6) siPAK1-1 GACAUCCAACAGCCAGAAA (SEQ ID NO: 7) siPAKI-2 GAGAAAGAGCGGCCAGAGA (SEQ ID NO: 8) hPAK1-6 UACCAGCACUAUGAUUGGA (SEQ ID NO: 9) siPAK1-7 UCUGUAUACACACGGUCUG (SEQ ID NO: 10) (Nasoff et al. 2007 U.S. Patent Application Publication No. US20070128204 filed Dec. 1, 2006), and three siRNA oligos (PAK1_p1, PAK1_p2, and PAK1_p3) obtained from Qiagen (Table 1). These siRNAs were validated by Oiagen using RT-PCR and shown to provide ≧70% target gene mRNA knockdown. These siRNAs were 21 bp duplexes with symmetric 2 bp 3′ overhangs.

TABLE 1

| Duplex | | | |
|---|---|---|---|
| Name | Target | Antisense | Sense |
| PAKl_p1 | TCCACTGATTGCTGCAGCTAA | UUAGCUGCAGCAAUCAGUGga | CACUGAUUGCUGCAGCUAAtt |
| | (SEQ ID NO: 12) | (SEQ ID NO:14) | (SEQ ID NO: 15) |
| PAK1_p2 | TTGAAGAGAACTGCAACTGAA | UUCAGUUGCAGUUCUCUUCaa | GAAGAGAACUGCAACUGAAtt |
| | (SEQ ID NO: 13) | (SEQ ID NO: 16) | (SEQ ID NO: 17) |
| PAK1_p3 | ACCCTAAACCATGGTTCTAAA | UUUAGAACCAUGGUUUAGGgt | CCUAAACCAUGGUUCUAAAtt |
| | (SEQ ID NO: 18 | (SEQ ID NO: 19 | (SEQ ID NO: 20) |

B. DYRK3

Mammalian DYRK3 (REDK, hYAK3) is a MAPK-related protein kinase that can target Ser/Thr sites. DYRK3 can be activated by tyrosine (auto)phosphorylation at a conserved YXY motif (or loop) between consensus kinase subdomains VII and VIII. DYRK3 can be selectively expressed at high levels in hematopoietic cells of erythroid lineage (Geiger, J N et al. (2001) Blood 97: 901-910; Lord, K A et al. (2000) Blood 95: 2838-2846). Inhibition of DYRK3 in primary murine and human hematopoietic progenitor cells with an antisense oligonucleotide can affect the production of colony-forming units-erythroid (the penultimate progenitors of erythroblasts). DYRK3 activity can depend upon the presence of Tyr³³³ within its predicted (auto)phosphorylation loop, and loop acidification can be activating (Li, K. et al. (2002) J Biol Chem 49, 47052-47060). DYRK3 can act via mechanisms involving the kinase domain as well as the unique C-terminal domain-dependent to regulate CREB and CRE response pathways via routes that depend on PKA (Li, K. et al (2002) supra). Expression of DYRK3 in FDC hematopoietic progenitor cells can regulate apoptosis (Li, K. et al (2002) supra).

Inhibitors of DYRK3 that can be used in the methods and compositions of the present invention include quinoline inhibitors of DYRK3/hYAK3 (U.S. Pat. No. 7,087,758), YAK3/DYRK3 inhibitor GSK626616AC, (http://clinicaltrials.gov/show/NCT00443170), 3-carboxy quinoline derivatives DYKR3/YAK3 (Burgess et al. U.S. Patent Application Publication No. 20060106058), and three siRNA oligos (DYRK3_p1, DYRK3_p2, and DYRK3_p3) obtained from Qiagen (Table 2). These siRNAs were validated by Oiagen using RT-PCR and shown to provide ≧70% target gene mRNA knockdown. These siRNAs were 21 bp duplexes with symmetric 2 bp 3′ overhangs.

TABLE 2

| Duplex | | | |
|---|---|---|---|
| Name | Target | Antisense | Sense |
| DYRK3_p1 | AGCCAATAAGCTTAAAGCTAA | UUAGCUUUAAGCUUAUUGGct | CCAAUAAGCUUAAAGCUAAtt |
| | (SEQ ID NO: 21) | (SEQ ID NO: 22) | (SEQ ID NO: 23) |
| DYRK3_p2 | TCGACAGTACGTGGCCCTAAA | UUUAGGGCCACGUACUGUCga | GACAGUACGUGGCCCUAAAtt |
| | (SEQ ID NO: 24) | (SEQ ID NO: 25) | (SEQ ID NO: 26) |
| DYRK3_p3 | AACGGGTAGTTAATCCTGCAA | UUGCAGGAUUAACUACCCGtt | CGGGUAGUUAAUCCUGCAAtt |
| | (SEQ ID NO: 27) | (SEQ ID NO: 28) | (SEQ ID NO: 29) |

C. PTK9 (TWF1)

Twinfilin1 is composed of two ADF/cofilin-like (ADF-H) domains connected by a short linker region and followed by a 20 residues C-terminal tail. The two ADF-H domains are approximately 20% homologous to each other (Lappalainen et al., (1998) Mol. Biol. Cell 9: 1951-1959).

Human twinfilin was originally identified as a tyrosine kinase (Beeler et al., (1994) Mol. Cell. Biol. 14: 982-988), but studies have demonstrated that it has no kinase activity (Vartiainen et al., (2000) Mol. Cell. Biol. 20:1772-1783; Rohwer et al., (1999) Eur. J. Biochem. 263:518-525), and twinfilin lacks sequence homology to known protein kinases. Rather, twinfilin can bind actin-monomers (Goode et al., (1998) J. Cell Biol. 142:723-733; Vartiainen et al., (2000) supra; Wahlström et al., (2001) J. Cell Biol. 155:787-796). Information concerning the function of twinfilin has been derived in part from studies of homologs in yeast and Drosophila. Twinfilin appears to form a 1:1 complex with actin monomers. Twinfilin can efficiently sequester actin-monomer (Goode et al., (1998) supra; Vartiainen et al., (2000) supra; Wahlström et al., (2001) supra). Twinfilin can interact with ADP-actin-monomers and can inhibit their nucleotide exchange and filament assembly (Palmgren et al., (2001) J. Cell Biol. 155:251-260). Twinfilin may interact with newly depolymerized, assembly-incompetent ADP-actin-monomers.

Twinfilin can have a punctate cytoplasmic staining pattern and can localize to cellular processes containing actin monomers and filaments in cultured mammalian cells (Vartiainen et al., (2000) supra). Direct interactions between twinfilin and capping protein can mediate the localization of twinfilin to the sites of rapid actin filament assembly (Palmgren et al., (2001) supra).

Inhibitors of TWF1/PTK9 that can be used in the methods and compositions of the present invention include shRNAs, including Sigma TRC (The RNAi Consortium) #: TRCN0000011013; Clone ID: NM_{—}002822.3-907s1c1; Accession Number(s): NM_{—}198974.1, NM_{—}002822.3; CCGGGCCTGGATACACATGCAGTATCTCGAGATACTGCATGTGTATCCAGGCTTTTT (SEQ ID NO: 30); Sigma TRC # TRCN0000006364; Clone ID: NM_{—}002822.3-2014s1c1; Accession Number(s): NM_{—}198974.1, NM_{—}002822.3; CCGGCCGAGCAAATACTCAGATTTACTCGAGTAAATCTGAGTATTTGCTCGGTTTTT (SEQ ID NO: 31); Sigma TRC # TRCN0000006365; Clone ID: NM_{—}002822.3-364s1c1; Accession Number(s): NM_{—}198974.1, NM_{—}002822.3; CCGGCCAGGGATATGAATGGATATTCTCGAGAATATCCATTCATATCCCTGGTTTTT (SEQ ID NO: 32); Sigma TRC # TRCN0000006366; Clone ID: NM_{—}002822.3-474s1c1; Accession Number(s): NM_{—}198974.1, NM_{—}002822.3; CCGGGCCACATTAAAGATGAAGTATCTCGAGATACTTCATCTTTAATGTGGCTTTTT (SEQ ID NO: 33); Sigma TRC #: TRCN0000006367; Clone ID: NM_{—}002822.3-962s1c1; Accession Number(s): NM_{—}198974.1, NM_{—}002822.3; CCGGCGTCTGCTAGAAATTGTAGAACTCGAGTTCTACAATTTCTAGCAGACGTTTTT (SEQ ID NO: 34).

Inhibitors of PTK9/TWF1 that can be used in the methods and compositions of the present invention also include PTK9 Pre-design chimeric RNAi (Cat. # H00005756-R04, Abnova) and PTK9 validated Stealth™ DuoPak (Cat. # 12938068, Invitrogen).

D. GPRK2L (GRK4)

G-protein coupled receptor (GPCR) kinases (GRKs) are serine/threonine kinases that can be organized into three families (Penela et al., (2003) Cell Signal 15: 973-981). One family is the GRK4 family, which consists of GRK4, GRK5, and GRK6. Characteristics of the GRK4 subfamily include: a) membrane localization owing to palmitoylation on C-terminal cysteine residues (for GRK4/6) or interaction between negatively charged membrane phospholipids and a domain that is positively charged near the C terminus (GRK5), b) activation by phosphatidylinositol bisphosphate binding (to an N-terminal domain), and c) inhibition by calcium-sensor proteins, for example, calmodulin (Pronin et al., (1997) J. Biol. Chem. 272: 18273-18280; Pitcher et al., (1998) Annu. Rev. Biochem. 67: 653-692; Kohout and Lefkowitz, (2003) Mol. Pharmacol. 63: 9-18; Willets et al., (2003) Trends Pharmacol, Sci. 24: 626-633).

Four RNA splice variants have been identified for GRK4: α, β, γ, and δ (Premont et al., (1996) J. Biol. Chem. 271:6403-6410). GRK4α is the full-length version. GRK4β lacks sequence encoded by exon 2, which results in a 32-amino acid deletion that includes the phosphatidylinositol bisphosphate binding domain near the N terminus. GRK4γ lacks sequence encoded by exon 15, which results in a 46-amino acid deletion near the C terminus. GRK4δ, the shortest variant, lacks sequence encoded by both alternatively spliced exons.

GRKs play a role in GCPR desensitization. GPCRs can undergo desensitization upon activation by agonist; this process that can result in abatement of receptor response under continued agonist stimulation (Ferguson et al., (1996) Can. J. Physiol. Pharmacol. 74: 1095-1110; Gainetdinov et al., (2004) Annu. Rev. Neurosci. 27: 107-144). GRK-mediated phosphorylation can decrease receptor/G protein interactions and initiate arrestin binding. Arrestin association can further decrease G protein coupling and enhance endocytosis of the receptor. GPCRs that are internalized can engage additional signaling pathways, be sorted for recycling to the plasma membrane, or be targeted for degradation (Ferguson et al., (1996) Can. J. Physiol. Pharmacol. 74: 1095-1110; Penela et al., (2003) Cell Signal 15: 973-981; Gainetdinov et al., (2004) Annu. Rev. Neurosci. 27: 107-144).

GRK4 can also stimulate agonist-independent phosphorylation of GPCRs. For example, GRK4 coexpression with the D1 receptor resulted in phosphorylation of the receptor that was only slightly increased upon addition of agonist (Rankin et al. (2006) Mol. Pharmacol. 69:759-769). Phosphorylation of the D1 receptor by GRK4α in the absence of agonist binding can result in reduced agonist-induced cAMP accumulation, an increase in basal receptor internalization, and reduced number of total receptors.

Inhibitors of GRK4 that can be used in the methods and compositions of the present invention include, for example, the antisense-oligonucleotide (As-Odn), 209 5′-CATGAAGTTCTC CAGTTCCAT-3′ 189 (SEQ ID NO: 19) (Sanada et al. (2006) Hypertension 47:1131-1139), calmodulin (Iacovelli et al. (1999) FASEB J. 13:1-8), heparin (an inhibitor of GRK4a; Sallese et al. (1997) J. Biol. Chem. 272:10188-10198), and three siRNA oligos (GPRK2L_p1, GPRK2L_p2, and GPRK2L_p3) obtained from Qiagen (Table 3). These siRNAs were validated by Oiagen using RT-PCR and shown to provide ≧70% target gene mRNA knockdown. These siRNAs were 21 bp duplexes with symetric 2 bp 3′ overhangs.

TABLE 3

| Duplex | | | |
|---|---|---|---|
| Name | Target | Antisense | Sense |
| GPRK2L_p1 | CAGGATGTTACTCACCAAGAA | UUCUUGGUGAGUAACAUCCtg | GGAUGUUACUCACCAAGAAtt |
| | (SEQ ID NO: 35) | (SEQ ID NO: 36) | (SEQ ID NO: 37) |
| GPRK2L_p2 | CCGGGTGTTTCAAAGACATCA | UGAUGUCUUUGAAACACCCgg | GGGUGUUUCAAAGACAUCAtt |
| | (SEQ ID NO: 38) | (SEQ ID NO: 39) | (SEQ ID NO: 40) |
| GPRK2L_p3 | CTCGGTGGTGAAAGGGATCTA | UAGAUCCCUUUCACCACCGag | CGGUGGUGAAAGGGAUCUAtt |
| | (SEQ ID NO: 41) | (SEQ ID NO: 42) | (SEQ ID NO: 43) |

E. Rac1

Rac1 is a small signaling G protein that is a member of the Rho family of GTPases. Rac1 is a target of PAK1. Inhibitors of Rac1 that can be used in the methods and compositions of the present invention include, for example, Rac1 inhibitor W56 (MVDGKPVNLGLWDTAG; (SEQ ID NO: 44); Cat. No. 2221; Tocris bioscience), Rac1 inhibitor (Cat. No. 553502; Calbiochem), Rac1 inhibitor NSC 23766, N6-[2-[[4-(Diethylamino)-1-methylbutyl]amino]-6-methyl-4-pyrimidinyl]-2-methyl-4,6-quinolinediamine trihydrochloride (Cat. No. 2161; Tocris bioscience).

F. Cdc42

Cdc42 is a small GTPase of the Rho-subfamily that can regulate signaling pathways that control cell morphology, migration, endocytosis and cell cycle progression Inhibitors of Cdc42 that can be used in the methods and compositions of the present invention include, for example, secramine B (Pelish et al. (2006) Biochem. Pharmacol. 71:1720-1726); secramine A (Xu et al. (2006) Org Biomol Chem 4:4149-4157); and ACK42 (Nur-E-Kamal et al. (1999) Oncogene 18:7787-7793).

### VI. Transgenic Cells and Non-Human Mammals

Transgenic animal models, including recombinant and knock-out animals, can be generated from the host nucleic acids described herein. Exemplary transgenic non-human mammals include, but are not limited to, mice, rats, chickens, cows, and pigs. In certain examples, a transgenic non-human mammal has a knock-out of one or more of the target sequences associated with a kinase, and has a decreased viral susceptibility, for example infection by influenza or a poxvirus. Such knock-out animals are useful for studying the stages of viral infection and reducing the transmission of viruses from animals to humans. In addition, animal viruses that utilize the same targets provided herein can be analyzed in the animals.

Expression of the sequence used to knock-out or functionally delete the desired gene can be regulated by choosing the appropriate promoter sequence. For example, constitutive promoters can be used to ensure that the functionally deleted gene is never expressed by the animal. In contrast, an inducible promoter can be used to control when the transgenic animal does or does not express the gene of interest. Exemplary inducible promoters include tissue-specific promoters and promoters responsive or unresponsive to a particular stimulus (such as light, oxygen, chemical concentration), including the tetracycline/doxycycoine regulated promoters (TET-off, TET-on), ecdysone-inducible promoter, and the Cre/loxP recombinase system.

In one embodiment a transgenic mouse with a human kinase gene or a disrupted endogenous kinase gene, can be examined after exposure to various mammalian viruses, such as influenza or poxvirus. Comparison data can provide insight into the life cycles of the virus and related viruses. Moreover, knock-out animals (such as pigs) that are otherwise susceptible to an infection (for example influenza) can be made to determine the resistance to infection conferred by disruption of the gene.

In an alternative embodiment a transgenic pig with a human kinase gene or a disrupted endogenous kinase gene, can be produced and used as an animal model to determine susceptibility to viral infections including influenza or poxvirus infections. Transgenic animals, including methods of making and using transgenic animals, are described in various patents and publication, such as WO 01/43540; WO 02/19811; U.S. Pub. Nos: 2001-0044937 and 2002-0066117; and U.S. Pat. Nos. 5,859,308; 6,281,408; and 6,376,743; which are herein incorporated by reference.

### VII. Rational Design of Kinase Inhibitors

One aspect of the present invention relates to agents that modulate a protein kinase(s), e.g., protein kinase(s) involved in viral infection of host cells. In some embodiments the agent may be an antibody, an inorganic compound, an organic compound, a protein/peptide drug or a small molecule, such as an siRNA. Preferably, the agents can inhibit PAK1, Cdc42, Rac1, DYRK3, PTK9, and GPRK2L. In some embodiments, such agents exert anti-viral effects in vitro and in vivo.

Still another aspect of the present invention relates to methods of obtaining and/or making a composition for inhibiting a host kinase by designing an inhibitor agent; testing whether the agent inhibits a host kinase; and using the agent in making a composition for inhibiting a host kinase. In some embodiments, the invention relates to methods for designing and testing agents that are kinase modulators and are capable of inhibiting more than one host kinase.

The rational design methods of the present invention are aided by the current understanding of the structures of PAK1, Cdc42, Rac1, DYRK3, PTK9, and GPRK2L. Preferably X-ray structures of the kinases are used to examine the binding of a test inhibitor agent to a kinase. There typically is a direct correlation between the “tightness” of binding of a candidate agent to the enzyme and the in vitro cellular activity of the agent.

In embodiments where the agent is an inorganic or organic compound, said compound can be designed and tested entirely using computational methods or a portion of such designing and testing can be done computationally and the remainder done with wet lab techniques.

Lead compounds that inhibit protein kinases involved in viral infection of host cells can be identified using a variety of methods. In one embodiment lead compounds are designed to inhibit target host cell kinases using computer assisted “in silico” methodology. Chemogenomic tools such as the Kinase Toolkit™ can be used to design ATP site-directed kinase inhibitors using a combination of bioinformatics, medicinal chemistry and computational knowledge resources. Modeling and display techniques are used to enhance this information through superposition of X-ray crystal structures and sub-pocket similarity analysis. The vast majority of known kinase inhibitors are ATP competitive, targeting the binding site within the catalytic domain. However, useful inhibitors can also occupy regions of the binding site not occupied by bound ATP. Inspection of available crystal structures from the Protein Data Bank has led to the observation that the activated or partially activated conformation of the ATP site around a bound inhibitor is broadly the same for all kinases (Birault 2006). Further, it is evident that a consistent limited number of primary residues are involved in small molecule binding. Phylogenetically different kinases such as p38α and FGF-1 exhibit tertiary structural commonality in the ATP binding site. This structural similarity provides a useful basis to identify novel kinase inhibitors that inhibit specific individual kinases as well as larger classes of structurally related kinases.

In another embodiment lead compounds are discovered using computational filters to identify lead compounds from databases of known compounds. Some of these databases may contain millions of compounds. The filters are designed to incorporate appropriate ADMET (adsorption, distribution, metabolism, excretion, toxicity) properties. These filters for medicinal chemistry tractibility are based on lists of desired chemical features. ADMET modeling can be used during compound optimization to define an acceptable property space that contains compounds likely to have the desired properties. In some embodiments more than one computation filter is applied to the analysis of known compounds. Applicable filters include, but are not limited to the Lipinski filter (rule of 5), the Veber (rule of 2) filter, ChemGPS, MDDR filter, Shoichet's Aggregators, Martin filter, Ghose filter, Egan filter, MedChem tractibility filter, Lead likeness, Caco-2 permeation filter and the Muegge filter. These filters can be configured to screen for any compound with desired properties, such as aqueous solubility, molecular weight, SlogP, and number of H-bond donors or acceptors, amongst others.

In an alternative embodiment libraries of agents, such as inorganic or organic compounds, which are known or are predicted to inhibit a particular family of kinases, will be tested for their ability to inhibit viral infection using the same system used to identify host cell proteins that modulate viral infection. In some embodiments the screen is carried out in a similar fashion, wherein the library of siRNAs is replaced with a library of compounds. The results of the chemical screening will be compared with siRNA screening results for each respective virus providing a rank ordered list of compounds. In some further embodiments in vitro enzyme assays will be performed on the top ordered hits of compounds, for example on the top 5, 10, 15, 20 or 25 compounds which demonstrated an ability to inhibit viral infection in the compound screen. Wherein, top compounds will be profiled for their ability to inhibit a host cell target kinase, or a kinase upstream or downstream of in a kinase signaling pathway. In some further embodiments top compounds which show the greatest efficacy at inhibiting viral infection and/or specificity of host cell kinase targeting will be tested for toxicity and in vivo efficacy using animal models of viral infection.

In some embodiments agents are identified or developed that target specific kinases, such as PAK1, DYRK3, PTK9, and GPRK2L, or a kinase or another entity upstream or downstream of PAK1, DYRK3, PTK9, and GPRK2L in a kinase signaling pathway.

Testing involves evaluation of the designed agents for inhibitory activity towards a host cell kinase. In some embodiments, the collection of designed agents may be evaluated by computational methods to predict their activity in inhibiting a host cell kinase, without physically synthesizing the agents. Such computational methods may also be used to predict other properties of the agents, such as solubility, membrane penetrability, metabolism and toxicity.

In some embodiments, testing involves synthesizing the designed agents and evaluating their activity in inhibiting a host cell kinase and/or to inhibit viral infection in one or more biological assays via wet lab techniques.

The activity of the synthesized agent can then be evaluated by a biological assay, which directly or indirectly reflects the inhibition of a host cell kinase, and/or the inhibition of a viral infection. Representative biological assays include, but are not limited to: 1) cell-free studies of kinase inhibition; 2) cell-free studies of viral inhibition; 3) whole-cell studies of inhibition of viral infection (such as viral transmission, entry, replication, biosynthesis, assembly, or exit); and 4) in vivo animal models of efficacy against viral infection, such as mouse, avian, primate or pig models infected with a specific virus.

With respect to in vitro assays, the ability of a candidate agent to inhibit a host cell kinase can be evaluated by contacting the agent with an assay mixture for measuring activity of a host cell kinase, and determining the activity of the enzyme in the presence and absence of the agent. A decrease in activity of a host cell kinase in the presence as opposed to the absence of the agent indicates a host cell kinase inhibitor.

An example of a cell-free host cell kinase assay involves that described in Clerk and Sugden, FEBS Letters, 426:93-96 (1998), incorporated herein by reference. Another exemplary system is the AMBIT platform (Kinomescan), a kinase profiling technology. The platform can be used to identify molecular interactions and determine specificity based on quantitatively measuring the binding of unlinked small molecules to the ATP sites of multiple kinases. For example the platform can be used to analyze inhibitors, revealing how tightly the agents bind to their intended kinase targets compared to other ‘off-target’ kinases. This ‘off-target’ binding can be used to identify side-effects of the inhibitors or may justify evaluating certain inhibitors for other viruses.

Animal models used to reflect responses to viral infections can be utilized to evaluate host cell kinase inhibitory activity in vivo. Exemplary animal models include, but are not limited to, mice, rats, ferrets, guinea pigs, pigs (Sus scrofa), horses, primates, and horses.

In some embodiments, the activity or potency of an agent is similar towards multiple host kinases, as measured by whole cell and/or in vivo assays of IC50 or ED50 values, as described in more detail below. In some embodiments, potencies of a single agent with respect to a multiple host cell kinases differ by no more than a factor of about 1000. In some further embodiments, potencies differ by no more than a factor of about 100. In some further particular embodiments, potencies differ by no more than a factor of about 10.

### VIII. Methods of Treatment

One embodiment of the present invention relates to methods of using pharmaceutical compositions and kits comprising agents that inhibit a kinase or kinases to inhibit or decrease a viral infection. Another embodiment of the present invention provides methods, pharmaceutical compositions, and kits for the treatment of animal subjects. The term “animal subject” as used herein includes humans as well as other mammals. The term “treating” as used herein includes achieving a therapeutic benefit and/or a prophylactic benefit. By therapeutic benefit is meant eradication or amelioration of the underlying viral infection. Also, a therapeutic benefit is achieved with the eradication or amelioration of one or more of the physiological symptoms associated with the underlying viral infection such that an improvement is observed in the animal subject, notwithstanding the fact that the animal subject may still be afflicted with the underlying virus.

For embodiments where a prophylactic benefit is desired, a pharmaceutical composition of the invention may be administered to a patient at risk of developing viral infection such as influenza, or HIV, or to a patient reporting one or more of the physiological symptoms of a viral infection, even though a diagnosis of the condition may not have been made. Administration may prevent the viral infection from developing, or it may reduce, lessen, shorten and/or otherwise ameliorate the viral infection that develops. The pharmaceutical composition may modulate a target kinase activity. Wherein, the term modulate includes inhibition of a target kinase or alternatively activation of a target kinase.

Reducing the activity of a protein kinase, is also referred to as “inhibiting” the kinase. The term “inhibits” and its grammatical conjugations, such as “inhibitory,” do not require complete inhibition, but refer to a reduction in kinase activity. In some embodiments such reduction is by at least 50%, at least 75%, at least 90%, and may be by at least 95% of the activity of the enzyme in the absence of the inhibitory effect, e.g., in the absence of an inhibitor. Conversely, the phrase “does not inhibit” and its grammatical conjugations refer to situations where there is less than 20%, less than 10%, and may be less than 5%, of reduction in enzyme activity in the presence of the agent. Further the phrase “does not substantially inhibit” and its grammatical conjugations refer to situations where there is less than 30%, less than 20%, and in some embodiments less than 10% of reduction in enzyme activity in the presence of the agent.

Increasing the activity of a protein kinase, is also referred to as “activating” the kinase. The term “activated” and its grammatical conjugations, such as “activating,” do not require complete activation, but refer to an increase in kinase activity. In some embodiments such increase is by at least 50%, at least 75%, at least 90%, and may be by at least 95% of the activity of the enzyme in the absence of the activation effect, e.g., in the absence of an activator. Conversely, the phrase “does not activate” and its grammatical conjugations refer to situations where there is less than 20%, less than 10%, and may be less than 5%, of an increase in enzyme activity in the presence of the agent. Further the phrase “does not substantially activate” and its grammatical conjugations refer to situations where there is less than 30%, less than 20%, and in some embodiments less than 10% of an increase in enzyme activity in the presence of the agent.

The ability to reduce enzyme activity is a measure of the potency or the activity of an agent, or combination of agents, towards or against the enzyme. Potency may be measured by cell free, whole cell and/or in vivo assays in terms of IC50, Kᵢ and/or ED50 values. An IC50 value represents the concentration of an agent required to inhibit enzyme activity by half (50%) under a given set of conditions. A Kᵢ value represents the equilibrium affinity constant for the binding of an inhibiting agent to the enzyme. An ED50 value represents the dose of an agent required to effect a half-maximal response in a biological assay. Further details of these measures will be appreciated by those of ordinary skill in the art, and can be found in standard texts on biochemistry, enzymology, and the like.

The present invention also includes kits that can be used to treat viral infection. These kits comprise an agent or combination of agents that inhibits a kinase or kinases and in some embodiments instructions teaching the use of the kit according to the various methods and approaches described herein. Such kits may also include information, such as scientific literature references, package insert materials, clinical trial results, and/or summaries of these and the like, which indicate or establish the activities and/or advantages of the agent. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like.

A. siRNA Therapeutics.

Double stranded oligonucleotides are formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises nucleotide sequence that is homologous to the target nucleic acid sequence.

Double stranded RNA induced gene silencing can occur on at least three different levels: (i) transcription inactivation, which refers to RNA guided DNA or histone methylation; (ii) siRNA induced mRNA degradation; and (iii) mRNA induced transcriptional attenuation. It is generally considered that the major mechanism of RNA induced silencing (RNA interference, or RNAi) in mammalian cells is mRNA degradation. RNA interference (RNAi) is a mechanism that inhibits gene expression at the stage of translation or by hindering the transcription of specific genes. Specific RNAi pathway proteins are guided by the dsRNA to the targeted messenger RNA (mRNA), where they “cleave” the target, breaking it down into smaller portions that can no longer be translated into protein. Initial attempts to use RNAi in mammalian cells focused on the use of long strands of dsRNA. However, these attempts to induce RNAi met with limited success, due in part to the induction of the interferon response, which results in a general, as opposed to a target-specific, inhibition of protein synthesis. Thus, long dsRNA is not a viable option for RNAi in mammalian systems. Another outcome is epigenetic changes to a gene-histone modification and DNA methylation-affecting the degree the gene is transcribed.

More recently it has been shown that when short (18-30 bp) RNA duplexes are introduced into mammalian cells in culture, sequence-specific inhibition of target mRNA can be realized without inducing an interferon response. Certain of these short dsRNAs, referred to as small inhibitory RNAs (“siRNAs”), can act catalytically at sub-molar concentrations to cleave greater than 95% of the target mRNA in the cell. A description of the mechanisms for siRNA activity, as well as some of its applications are described in Provost et al., Ribonuclease Activity and RNA Binding of Recombinant Human Dicer, E.M.B.O. J., 2002 Nov. 1; 21(21): 5864-5874; Tabara et al., The dsRNA Binding Protein RDE-4 Interacts with RDE-1, DCR-1 and a DexH-box Helicase to Direct RNAi in C. elegans, Cell 2002, June 28; 109(7):861-71; Ketting et al., Dicer Functions in RNA Interference and in Synthesis of Small RNA Involved in Developmental Timing in C. elegans; Martinez et al., Single-Stranded Antisense siRNAs Guide Target RNA Cleavage in RNAi, Cell 2002, Sep. 6; 110(5):563; Hutvagner & Zamore, A microRNA in a multiple-turnover RNAi enzyme complex, Science 2002, 297:2056.

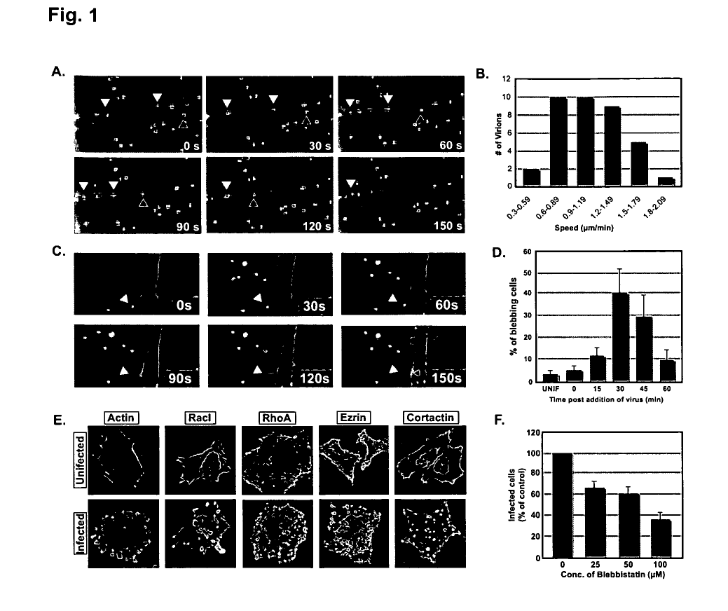

From a mechanistic perspective, introduction of long double stranded RNA into plants and invertebrate cells is broken down into siRNA by a Type III endonuclease known as Dicer. Sharp, RNA interference—2001, Genes Dev. 2001, 15:485. Dicer, a ribonuclease-III-like enzyme, processes the dsRNA into 19-23 base pair short interfering RNAs with characteristic two base 3′ overhangs. Bernstein, Caudy, Hammond, & Hannon, Role for a bidentate ribonuclease in the initiation step of RNA interference, Nature 2001, 409:363. The siRNAs are then incorporated into an RNA-induced silencing complex (RISC) where one or more helicases unwind the siRNA duplex, enabling the complementary antisense strand to guide target recognition. Nykanen, Haley, & Zamore, ATP requirements and small interfering RNA structure in the RNA interference pathway, Cell 2001, 107:309. Upon binding to the appropriate target mRNA, one or more endonucleases within the RISC cleaves the target to induce silencing. Elbashir, Lendeckel, & Tuschl, RNA interference is mediated by 21- and 22-nucleotide RNAs, Genes Dev 2001, 15:188, FIG. 1.

Generally, the antisense sequence is retained in the active RISC complex and guides the RISC to the target nucleotide sequence by means of complementary base-pairing of the antisense sequence with the target sequence for mediating sequence-specific RNA interference. It is known in the art that in some cell culture systems, certain types of unmodified siRNAs can exhibit “off target” effects. It is hypothesized that this off-target effect involves the participation of the sense sequence instead of the antisense sequence of the siRNA in the RISC complex (see for example Schwarz et al., 2003, Cell, 115, 199-208). In this instance the sense sequence is believed to direct the RISC complex to a sequence (off-target sequence) that is distinct from the intended target sequence, resulting in the inhibition of the off-target sequence In these double stranded nucleic acid molecules, each strand is complementary to a distinct target nucleic acid sequence. However, the off-targets that are affected by these dsRNAs are not entirely predictable and are non-specific.

The term “siRNA” refers to small inhibitory RNA duplexes that induce the RNA interference (RNAi) pathway. These molecules can vary in length (generally between 18-30 basepairs) and contain varying degrees of complementarity to their target mRNA in the antisense strand. Some, but not all, siRNA have unpaired overhanging bases on the 5′ or 3′ end of the sense strand and/or the antisense strand. The term “siRNA” includes duplexes of two separate strands, as well as single strands that can form hairpin structures comprising a duplex region. Small interfering RNA (siRNA), sometimes known as short interfering RNA or silencing RNA, are a class of 20-25 nucleotide-long double-stranded RNA molecules that play a variety of roles in biology.

While the two RNA strands do not need to be completely complementary, the strands should be sufficiently complementary to hybridize to form a duplex structure. In some instances, the complementary RNA strand may be less than 30 nucleotides, preferably less than 25 nucleotides in length, more preferably 19 to 24 nucleotides in length, more preferably 20-23 nucleotides in length, and even more preferably 22 nucleotides in length. The dsRNA of the present invention may further comprise at least one single-stranded nucleotide overhang. The dsRNA of the present invention may further comprise a substituted or chemically modified nucleotide. As discussed in detail below, the dsRNA can be synthesized by standard methods known in the art.

SiRNA may be divided into five (5) groups (non-functional, semi-functional, functional, highly functional, and hyper-functional) based on the level or degree of silencing that they induce in cultured cell lines. As used herein, these definitions are based on a set of conditions where the siRNA is transfected into said cell line at a concentration of 100 nM and the level of silencing is tested at a time of roughly 24 hours after transfection, and not exceeding 72 hours after transfection. In this context, “non-functional siRNA” are defined as those siRNA that induce less than 50% (<50%) target silencing. “Semi-functional siRNA” induce 50-79% target silencing. “Functional siRNA” are molecules that induce 80-95% gene silencing. “Highly-functional siRNA” are molecules that induce greater than 95% gene silencing. “Hyperfunctional siRNA” are a special class of molecules. For purposes of this document, hyperfunctional siRNA are defined as those molecules that: (1) induce greater than 95% silencing of a specific target when they are transfected at subnanomolar concentrations (i.e., less than one nanomolar); and/or (2) induce functional (or better) levels of silencing for greater than 96 hours. These relative functionalities (though not intended to be absolutes) may be used to compare siRNAs to a particular target for applications such as functional genomics, target identification and therapeutics.

A. MicroRNA Therapeutics

microRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression.

### IX. Formulations, Routes of Administration, and Effective Doses

Yet another aspect of the present invention relates to formulations, routes of administration and effective doses for pharmaceutical compositions comprising an agent or combination of agents of the instant invention. Such pharmaceutical compositions can be used to treat viral infections as described above.

The agents or their pharmaceutically acceptable salts may be provided alone or in combination with one or more other agents or with one or more other forms. For example a formulation may comprise one or more agents in particular proportions, depending on the relative potencies of each agent and the intended indication. For example, in compositions for targeting two different host targets, and where potencies are similar, about a 1:1 ratio of agents may be used. The two forms may be formulated together, in the same dosage unit e.g. in one cream, suppository, tablet, capsule, aerosol spray, or packet of powder to be dissolved in a beverage; or each form may be formulated in a separate unit, e.g., two creams, two suppositories, two tablets, two capsules, a tablet and a liquid for dissolving the tablet, two aerosol sprays, or a packet of powder and a liquid for dissolving the powder, etc.

The term “pharmaceutically acceptable salt” means those salts which retain the biological effectiveness and properties of the agents used in the present invention, and which are not biologically or otherwise undesirable. For example, a pharmaceutically acceptable salt does not interfere with the beneficial effect of a agent of the invention in inhibiting a kinase, such as a kinase selected from the group consisting of PAK1, DYRK3, PTK9, and GPRK2L.

Typical salts are those of the inorganic ions, such as, for example, sodium, potassium, calcium, magnesium ions, and the like. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the agent(s) contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine, triethanolamine, and the like.

A pharmaceutically acceptable ester or amide refers to those which retain biological effectiveness and properties of the agents used in the present invention, and which are not biologically or otherwise undesirable. For example, the ester or amide does not interfere with the beneficial effect of an agent of the invention in inhibiting a kinase, such as a kinase selected from the group consisting of: PAK1, DYRK3, PTK9, and GPRK2L. Typical esters include ethyl, methyl, isobutyl, ethylene glycol, and the like. Typical amides include unsubstituted amides, alkyl amides, dialkyl amides, and the like.

In some embodiments, an agent may be administered in combination with one or more other compounds, forms, and/or agents, e.g., as described above. Pharmaceutical compositions comprising combinations of a kinase inhibitor with one or more other active agents can be formulated to comprise certain molar ratios. For example, molar ratios of about 99:1 to about 1:99 of a kinase inhibitor to the other active agent can be used. In some subset of the embodiments, the range of molar ratios of kinase inhibitor: other active agent is selected from about 80:20 to about 20:80; about 75:25 to about 25:75, about 70:30 to about 30:70, about 66:33 to about 33:66, about 60:40 to about 40:60; about 50:50; and about 90:10 to about 10:90. The molar ratio may of kinase inhibitor: other active agent may be about 1:9, and in some embodiments may be about 1:1. The two agents, forms and/or compounds may be formulated together, in the same dosage unit e.g. in one cream, suppository, tablet, capsule, or packet of powder to be dissolved in a beverage; or each agent, form, and/or compound may be formulated in separate units, e.g., two creams, suppositories, tablets, two capsules, a tablet and a liquid for dissolving the tablet, an aerosol spray a packet of powder and a liquid for dissolving the powder, etc.

If necessary or desirable, the agents and/or combinations of agents may be administered with still other agents. The choice of agents that can be co-administered with the agents and/or combinations of agents of the instant invention can depend, at least in part, on the condition being treated. Agents of particular use in the formulations of the present invention include, for example, any agent having a therapeutic effect for a viral infection, including, e.g., drugs used to treat inflammatory conditions. For example, in treatments for influenza, in some embodiments formulations of the instant invention may additionally contain one or more conventional anti-inflammatory drugs, such as an NSAID, e.g. ibuprofen, naproxen, acetominophen, ketoprofen, or aspirin. In some alternative embodiments for the treatment of influenza formulations of the instant invention may additionally contain one or more conventional influenza antiviral agents, such as amantadine, rimantadine, zanamivir, and oseltamivir. In treatments for retroviral infections, such as HIV, formulations of the instant invention may additionally contain one or more conventional antiviral drug, such as protease inhibitors (lopinavir/ritonavir {Kaletra}, indinavir {Crixivan}, ritonavir {Norvir}, nelfinavir {Viracept}, saquinavir hard gel capsules {Invirase}, atazanavir {Reyataz}, amprenavir {Agenerase}, fosamprenavir {Telzir}, tipranavir{Aptivus}), reverse transcriptase inhibitors, includingnon-Nucleoside and Nucleoside/nucleotide inhibitors (AZT {zidovudine, Retrovir}, ddI {didanosine, Videx}, 3TC {lamivudine, Epivir}, d4T {stavudine, Zerit}, abacavir {Ziagen}, FTC {emtricitabine, Emtriva}, tenofovir {Viread}, efavirenz {Sustiva} and nevirapine {Viramune}), fusion inhibitors T20 {enfuvirtide, Fuzeon}, integrase inhibitors (MK-0518 and GS-9137), and maturation inhibitors (PA-457 {Bevirimat}). As another example, formulations may additionally contain one or more supplements, such as vitamin C, E or other anti-oxidants.

The agent(s) (or pharmaceutically acceptable salts, esters or amides thereof) may be administered per se or in the form of a pharmaceutical composition wherein the active agent(s) is in an admixture or mixture with one or more pharmaceutically acceptable carriers. A pharmaceutical composition, as used herein, may be any composition prepared for administration to a subject. Pharmaceutical compositions for use in accordance with the present invention may be formulated in conventional manner using one or more physiologically acceptable carriers, comprising excipients, diluents, and/or auxiliaries, e.g., which facilitate processing of the active agents into preparations that can be administered. Proper formulation may depend at least in part upon the route of administration chosen. The agent(s) useful in the present invention, or pharmaceutically acceptable salts, esters, or amides thereof, can be delivered to a patient using a number of routes or modes of administration, including oral, buccal, topical, rectal, transdermal, transmucosal, subcutaneous, intravenous, and intramuscular applications, as well as by inhalation.

For oral administration, the agents can be formulated readily by combining the active agent(s) with pharmaceutically acceptable carriers well known in the art. Such carriers enable the agents of the invention to be formulated as tablets, including chewable tablets, pills, dragees, capsules, lozenges, hard candy, liquids, gels, syrups, slurries, powders, suspensions, elixirs, wafers, and the like, for oral ingestion by a patient to be treated. Such formulations can comprise pharmaceutically acceptable carriers including solid diluents or fillers, sterile aqueous media and various non-toxic organic solvents. Generally, the agents of the invention will be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

Aqueous suspensions for oral use may contain agent(s) of this invention with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like.

In some embodiments, oils or non-aqueous solvents may be required to bring the agents into solution, due to, for example, the presence of large lipophilic moieties. Alternatively, emulsions, suspensions, or other preparations, for example, liposomal preparations, may be used. With respect to liposomal preparations, any known methods for preparing liposomes for treatment of a condition may be used. See, for example, Bangham et al., J. Mol. Biol. 23: 238-252 (1965) and Szoka et al., Proc. Natl. Acad. Sci. USA 75: 4194-4198 (1978), incorporated herein by reference. Ligands may also be attached to the liposomes to direct these compositions to particular sites of action. Agents of this invention may also be integrated into foodstuffs, e.g., cream cheese, butter, salad dressing, or ice cream to facilitate solubilization, administration, and/or compliance in certain patient populations.

Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; flavoring elements, cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate. The agents may also be formulated as a sustained release preparation.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for administration.

For injection, the agents of the present invention may be formulated in aqueous solutions, including but not limited to physiologically compatible buffers such as Hank's solution, Ringer's solution, or physiological saline buffer. Such compositions may also include one or more excipients, for example, preservatives, solubilizers, fillers, lubricants, stabilizers, albumin, and the like. Methods of formulation are known in the art, for example, as disclosed in Remington's Pharmaceutical Sciences, latest edition, Mack Publishing Co., Easton P.

In addition to the formulations described previously, the agents may also be formulated as a depot preparation. Such long acting formulations may be administered by implantation or transcutaneous delivery (for example subcutaneously or intramuscularly), intramuscular injection or use of a transdermal patch. Thus, for example, the agents may be formulated with suitable polymeric or hydrophobic materials (for example as an emulsion in an acceptable oil) or ion exchange resins, or as sparingly soluble derivatives, for example, as a sparingly soluble salt.

In some embodiments, pharmaceutical compositions comprising one or more agents of the present invention exert local and regional effects when administered topically or injected at or near particular sites of infection. Direct topical application, e.g., of a viscous liquid, gel, jelly, cream, lotion, ointment, suppository, foam, or aerosol spray, may be used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations may also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983. In some embodiments, local/topical formulations comprising a kinase inhibitor are used to treat epidermal or mucosal viral infections.

Pharmaceutical compositions of the present invention may contain a cosmetically or dermatologically acceptable carrier. Such carriers are compatible with skin, nails, mucous membranes, tissues and/or hair, and can include any conventionally used cosmetic or dermatological carrier meeting these requirements. Such carriers can be readily selected by one of ordinary skill in the art. In formulating skin ointments, an agent or combination of agents of the instant invention may be formulated in an oleaginous hydrocarbon base, an anhydrous absorption base, a water-in-oil absorption base, an oil-in-water water-removable base and/or a water-soluble base.

The compositions according to the present invention may be in any form suitable for topical application, including aqueous, aqueous-alcoholic or oily solutions, lotion or serum dispersions, aqueous, anhydrous or oily gels, emulsions obtained by dispersion of a fatty phase in an aqueous phase (O/W or oil in water) or, conversely, (W/O or water in oil), microemulsions or alternatively microcapsules, microparticles or lipid vesicle dispersions of ionic and/or nonionic type. These compositions can be prepared according to conventional methods. Other than the agents of the invention, the amounts of the various constituents of the compositions according to the invention are those conventionally used in the art. These compositions in particular constitute protection, treatment or care creams, milks, lotions, gels or foams for the face, for the hands, for the body and/or for the mucous membranes, or for cleansing the skin. The compositions may also consist of solid preparations constituting soaps or cleansing bars.

Compositions of the present invention may also contain adjuvants common to the cosmetic and dermatological fields, such as hydrophilic or lipophilic gelling agents, hydrophilic or lipophilic active agents, preserving agents, antioxidants, solvents, fragrances, fillers, sunscreens, odor-absorbers and dyestuffs. The amounts of these various adjuvants are those conventionally used in the fields considered and, for example, are from about 0.01% to about 20% of the total weight of the composition. Depending on their nature, these adjuvants may be introduced into the fatty phase, into the aqueous phase and/or into the lipid vesicles.

In some embodiments, ocular viral infections can be effectively treated with ophthalmic solutions, suspensions, ointments or inserts comprising an agent or combination of agents of the present invention.

In some embodiments, viral infections of the ear can be effectively treated with otic solutions, suspensions, ointments or inserts comprising an agent or combination of agents of the present invention.

In some embodiments, the agents of the present invention are delivered in soluble rather than suspension form, which allows for more rapid and quantitative absorption to the sites of action. In general, formulations such as jellies, creams, lotions, suppositories and ointments can provide an area with more extended exposure to the agents of the present invention, while formulations in solution, e.g., sprays, provide more immediate, short-term exposure.

In some embodiments relating to topical/local application, the pharmaceutical compositions can include one or more penetration enhancers. For example, the formulations may comprise suitable solid or gel phase carriers or excipients that increase penetration or help delivery of agents or combinations of agents of the invention across a permeability barrier, e.g., the skin. Many of these penetration-enhancing compounds are known in the art of topical formulation, and include, e.g., water, alcohols (e.g., terpenes like methanol, ethanol, 2-propanol), sulfoxides (e.g., dimethyl sulfoxide, decylmethyl sulfoxide, tetradecylmethyl sulfoxide), pyrrolidones (e.g., 2-pyrrolidone, N-methyl-2-pyrrolidone, N-(2-hydroxyethyl)pyrrolidone), laurocapram, acetone, dimethylacetamide, dimethylformamide, tetrahydrofurfuryl alcohol, L-α-amino acids, anionic, cationic, amphoteric or nonionic surfactants (e.g., isopropyl myristate and sodium lauryl sulfate), fatty acids, fatty alcohols (e.g., oleic acid), amines, amides, clofibric acid amides, hexamethylene lauramide, proteolytic enzymes, a-bisabolol, d-limonene, urea and N,N-diethyl-m-toluamide, and the like. Additional examples include humectants (e.g., urea), glycols (e.g., propylene glycol and polyethylene glycol), glycerol monolaurate, alkanes, alkanols, ORGELASE, calcium carbonate, calcium phosphate, various sugars, starches, cellulose derivatives, gelatin, and/or other polymers. In some embodiments, the pharmaceutical compositions will include one or more such penetration enhancers.

In some embodiments, the pharmaceutical compositions for local/topical application can include one or more antimicrobial preservatives such as quaternary ammonium compounds, organic mercurials, p-hydroxy benzoates, aromatic alcohols, chlorobutanol, and the like.

Gastrointestinal viral infections can be effectively treated with orally- or rectally delivered solutions, suspensions, ointments, enemas and/or suppositories comprising an agent or combination of agents of the present invention.

Respiratory viral infections can be effectively treated with aerosol solutions, suspensions or dry powders comprising an agent or combination of agents of the present invention. Administration by inhalation is particularly useful in treating viral infections of the lung, such as influenza. The aerosol can be administered through the respiratory system or nasal passages. For example, one skilled in the art will recognize that a composition of the present invention can be suspended or dissolved in an appropriate carrier, e.g., a pharmaceutically acceptable propellant, and administered directly into the lungs using a nasal spray or inhalant. For example, an aerosol formulation comprising a kinase inhibitor can be dissolved, suspended or emulsified in a propellant or a mixture of solvent and propellant, e.g., for administration as a nasal spray or inhalant. Aerosol formulations may contain any acceptable propellant under pressure, such as a cosmetically or dermatologically or pharmaceutically acceptable propellant, as conventionally used in the art.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

An aerosol formulation for inhalations and inhalants can be designed so that the agent or combination of agents of the present invention is carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants useful in the present invention include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Halocarbon propellants are described in Johnson, U.S. Pat. No. 5,376,359, issued Dec. 27, 1994; Byron et al., U.S. Pat. No. 5,190,029, issued Mar. 2, 1993; and Purewal et al., U.S. Pat. No. 5,776,434, issued Jul. 7, 1998. Hydrocarbon propellants useful in the invention include, for example, propane, isobutane, n-butane, pentane, isopentane and neopentane. A blend of hydrocarbons can also be used as a propellant. Ether propellants include, for example, dimethyl ether as well as the ethers. An aerosol formulation of the invention can also comprise more than one propellant. For example, the aerosol formulation can comprise more than one propellant from the same class, such as two or more fluorocarbons; or more than one, more than two, more than three propellants from different classes, such as a fluorohydrocarbon and a hydrocarbon. Pharmaceutical compositions of the present invention can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

The aerosol formulation can be packaged under pressure and can be formulated as an aerosol using solutions, suspensions, emulsions, powders and semisolid preparations. For example, a solution aerosol formulation can comprise a solution of an agent of the invention such as a kinase inhibitor in (substantially) pure propellant or as a mixture of propellant and solvent. The solvent can be used to dissolve the agent and/or retard the evaporation of the propellant. Solvents useful in the invention include, for example, water, ethanol and glycols. Any combination of suitable solvents can be use, optionally combined with preservatives, antioxidants, and/or other aerosol components.

An aerosol formulation can also be a dispersion or suspension. A suspension aerosol formulation may comprise a suspension of an agent or combination of agents of the instant invention, e.g., a kinase inhibitor, and a dispersing agent. Dispersing agents useful in the invention include, for example, sorbitan trioleate, oleyl alcohol, oleic acid, lecithin and corn oil. A suspension aerosol formulation can also include lubricants, preservatives, antioxidant, and/or other aerosol components.

An aerosol formulation can similarly be formulated as an emulsion. An emulsion aerosol formulation can include, for example, an alcohol such as ethanol, a surfactant, water and a propellant, as well as an agent or combination of agents of the invention, e.g., a kinase inhibitor. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

Pharmaceutical compositions suitable for use in the present invention include compositions wherein the active ingredients are present in an effective amount, i.e., in an amount effective to achieve therapeutic and/or prophylactic benefit in a host with at least one viral infection. The actual amount effective for a particular application will depend on the condition or conditions being treated, the condition of the subject, the formulation, and the route of administration, as well as other factors known to those of skill in the art. Determination of an effective amount of a kinase inhibitor is well within the capabilities of those skilled in the art, in light of the disclosure herein, and will be determined using routine optimization techniques.

The effective amount for use in humans can be determined from animal models. For example, a dose for humans can be formulated to achieve circulating, liver, topical and/or gastrointestinal concentrations that have been found to be effective in animals. One skilled in the art can determine the effective amount for human use, especially in light of the animal model experimental data described herein. Based on animal data, and other types of similar data, those skilled in the art can determine the effective amounts of compositions of the present invention appropriate for humans.

The effective amount when referring to an agent or combination of agents of the invention will generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

Further, appropriate doses for kinase inhibitor can be determined based on in vitro experimental results. For example, the in vitro potency of an agent in inhibiting a kinase, such as PAK1, DYRK3, PTK9, and GPRK2L, provides information useful in the development of effective in vivo dosages to achieve similar biological effects.

In some embodiments, administration of agents of the present invention may be intermittent, for example administration once every two days, every three days, every five days, once a week, once or twice a month, and the like. In some embodiments, the amount, forms, and/or amounts of the different forms may be varied at different times of administration.

A person of skill in the art would be able to monitor in a patient the effect of administration of a particular agent. For example, HIV viral load levels can be determined by techniques standard in the art, such as measuring CD4 cell counts, and/or viral levels as detected by PCR. Other techniques would be apparent to one of skill in the art.

### Bioterrorism

The provided invention can be used to treat viral infections caused by a bioterrorist attack. Viruses that can be used in a bioterrorist attack include, for example, Variola major virus, which causes small pox; encephalitis viruses, such as western equine encephalitis virus, eastern equine encephalitis virus, and Venezuelan equine encephalitis virus, and arenaviruses (Lassa, Machupo), bunyaviruses, filoviruses (Ebola, Marburg), and flaviviruses, which cause hemorrhagic fever. The provided invention can be stockpiled for use in treating viral infections caused by a bioterrorist attack to strengthen the capacities for medical responses.

### EXAMPLES

### Example One

### Evaluation of Entry Mechanism of Vaccinia Virus

To study the entry of vaccinia virus into cells we first generated recombinant mature virus particles (MVs) in which one of the core proteins, A5, was tagged at its N-terminus with monomeric yellow fluorescent protein (mYFP-MV). When added to HeLa cells expressing EGFP-tagged actin, we observed many of the brightly fluorescent viruses bind to filopodia, and proceed to move (‘surf’), along the filopodia towards the cell body (FIG. 1A). The movement was generally smooth and uninterrupted with a rate approximating that of actin retrograde flow (˜1 μm/min; FIG. 1B). When the MVs reached the cell body, a dramatic change occurred: a large bleb extruded from the plasma membrane at the site of contact with the virus. The expansion lasted for 30+/−4 sec, and the bleb remained extended for 10+/−2 sec before it retracted with the virus (FIG. 1C). The blebbing peaked at 30 min after virus addition with 40% of cells showing one or more blebs (FIG. 1D).

Indirect immunofluorescense showed that in addition to actin-GFP, the blebs contained a variety of actin-associated proteins such as Rac1, RhoA, ezrin, and cortactin (FIG. 1E). Blebbistatin, a myosin II inhibitor (Limouze et al., 2004), prevented formation of the blebs and inhibited viral infection by 62% suggesting that bleb formation plays a role in productive entry (FIG. 1F). Similar results were seen in multiple HeLa and BSC40 green monkey kidney cell lines.

Not only did some of the viruses use filopodia to reach the cell body, but they triggered a signaling cascade that resulted in the formation of transient membrane blebs. The blebs resembled those observed during cell motility, cytokinesis, and apoptosis (Charras et al., 2006; Fishkind et al., 1991; Mills et al., 1998).

FIG. 1 depicts surfing and membrane perturbation during mature virion entry. FIG. 1A depicts surfing of MVs along filopodia. Recombinant A5-YFP MVs were added to HeLa cells expressing transiently transfected GFP-actin. Images were taken at 1 Hz for 2.5 min at 37° C. Time points correspond to real time images of virions. Arrows correspond to individual virions. FIG. 1B depicts determination of MV surfing speed. The speed of 36 individual virions was determined by the difference of distance traveled over time (μm/min). FIG. 1C depicts induction of membrane blebbing. Recombinant A5-YFP MVs were added to HeLa cells expressing GFP-actin and imaged at 1 Hz for 2.5 min at 37° C. Arrow indicates actin patch formation at site of bleb collapse. FIG. 1D depicts time course of MV induced cellular blebbing. MVs were bound to HeLa cells for 1 h at 4° C. Cells were washed and shifted to 37° C. for the indicated times prior to fixation in 4% FA. Fifty cells at each time point were scored for blebbing and as displayed as percent of cells blebbing relative to uninfected controls. Experiments were done in triplicate and results averaged. FIG. 1E depicts determination of cellular factors localizing to blebs. HeLa cells transiently transfected with the indicated fluorescently tagged proteins were left untreated or infected with MVs. Cells were fixed 30 minutes post infection (mpi) and analyzed by confocal microscopy. Ten Z-stakes per cell were collected and displayed as a Z-projection. FIG. 1F depicts blebbing and infectivity. HeLa cells were pretreated with varying concentrations of Blebbistatin prior to infection with recombinant MVs expressing EGFP from an early/late viral promoter (EGFP-MV). The percentage of infected cells was determined by FACS analysis. The percentage of infected cells is displayed relative to control infections. Experiments were done in triplicate and results averaged.

To identify specific cellular kinases involved, we next performed a screen using interfering (si) RNAs to silence 50 kinases in HeLa cells. After transfection with single siRNAs, recombinant MV's were added that expressed EGFP from an early/late promoter (EGFP-MV), and the cells were analyzed for EGFP expression after 12 h. Three different siRNAs were used for each gene, and the significance was set at a three-fold repression of EGFP signal compared to mock infected cells or cells transfected with control siRNAs. Of the 50 kinases, PAK1 was found to inhibit EGFP expression, which means that virus binding, entry, transcription, or translation of early genes was suppressed.

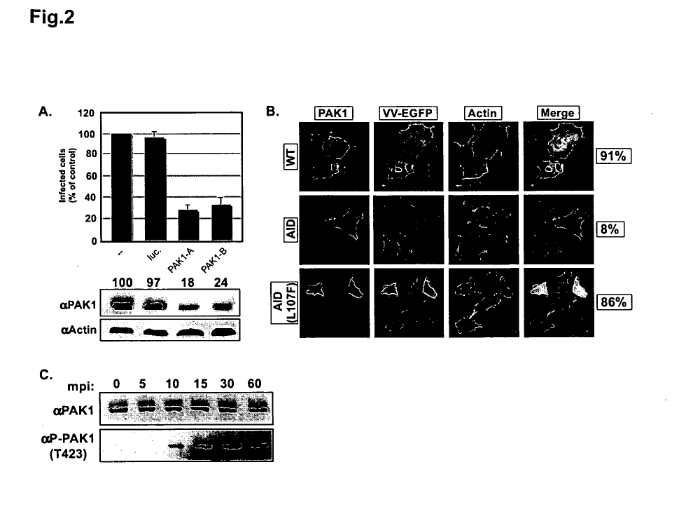

To validate the requirement of PAK1 in infection, we first used two additional siRNAs and found that these caused 82% and 76% knockdown of PAK1 as determined by immunoblotting (FIG. 2A; top). A fluorescence-activated cell-sorting (FACS) based infection assay showed that the number of infected cells was reduced by 74% and 68%, respectively (FIG. 2A; bottom). Previous reports have demonstrated that over-expression of a PAK1 domain comprising residues 83-149 (AID, the autoinhibitory domain) inhibits macropinocytosis (Dharmawardhane et al., 2000). In HeLa cells, we found that expression of the AID inhibited infection by 80% relative to cells over-expressing wt PAK1. Expression of a mutant form of AID (AID L107F) had no affect (FIG. 2B).

Phosphorylation of threonine residue 423 in PAK1 plays a role in activating macropinocytosis (Dharmawardhane et al., 2000). When MV's were added to cells, phosphorylated PAK1 was detected within 10 min and PAK1 remained activated over 60 min (FIG. 2C). A maximal response was seen with 30 mpi. The results were consistent with the time course of virion uptake and endosomal release of viral cores (Townsley et al., 2006). Taken together, the results demonstrated that PAK1 activity plays a role in productive entry of vaccinia MV's into cells. Evidently, the virus triggered the activation of Rac1, which in turn resulted in the activation of PAK1, and other downstream factors involved in actin dynamics.

FIG. 2 depicts p21-activated kinase-1 (PAK1) is required for MV entry. FIG. 2A. depicts the effect of siRNA knockdown of PAK1 on MV infection. HeLa cells were treated with two independently validated siRNAs (Qiagen; A:TCCACTGATTGCTGCAGCTAA (SEQ ID NO: 12); B:TTGAAGAGAACTGCAACTGAA (SEQ ID NO: 13) directed against PAK1. Thirty-six hours after treatment cells were infected with EGFP-MV at an MOI of 1 and harvested for analysis at 2 hpi. The percentage of infected cells was determined by FACS analysis. Experiments were performed in triplicate and results averaged Immunoblot analysis against PAK1 (a-PAK1; Santa Cruz) was performed to confirm the reduction of PAK1 protein levels (lower panel). A total of 50 μg of cell lysate was loaded per lane and the % of remaining PAK1 protein relative to mock treated cells (—) determined Immunblot against actin was utilized as a loading control. FIG. 2B depicts the effect of dominant-negative PAK1 on MV infectivity. HeLa cells were transiently transfected with fluorescent-tagged versions wild type PAK1 (WT), the PAK1 auto-inhibitory domain (AID), or a mutant version of the AID (AID L107F). Cells were infected with EGFP-MV at an MOI of 1. At 4 hpi cells were fixed and stained for actin. Cells were analyzed by confocal microscopy for transfected proteins (red), viral infection (green) and actin (blue). Experiments were performed in triplicate and 100 transfected cells per experiment scored for infection. Results are displayed as the average percentage of transfected/infected cells. FIG. 2C depicts activation of PAK1 during MV infection. MVs were bound to HeLa cells for 1 h at 4° C. Cells were washed 2× with cold PBS. Pre-warmed media was added and infections shifted to 37° C. prior to harvesting at the indicated time points. Immunoblot analysis for PAK1 and phosphorylated PAK1 (a-PAK1-Thr423; Cell Signaling) was performed.

It is known that PAK1 is involved in macropinocytosis, a ligand-induced, endocytic process that leads to the internalization of large amounts of fluid and plasma membrane in many different cell types (Falcone et al., 2006). Macropinocytosis is dependent on dynamic actin rearrangements, and it requires cholesterol as well as the small GTPases Rac1, CDC42, and Arf6 (Kirkham and Parton, 2005). In addition it involves Na⁺/H⁺ exchangers, tyrosine kinases, and at least one serine threonine kinase, PKC, in addition to PAK1 (Dharmawardhane et al., 2000; Grimmer et al., 2002; Hewlett et al., 1994; Veithen et al., 1996). Dynamin is not required (Damke et al., 1994).

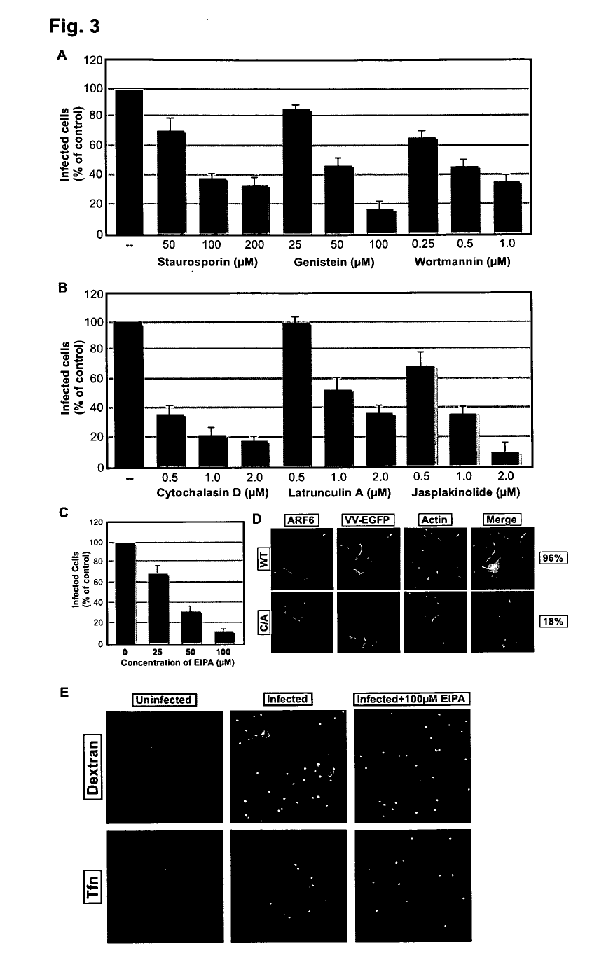

To test whether MV entry involved macropinocytosis, we tested a variety of broad-range kinase inhibitors. Staurosporin (serine/threonine kinase inhibitor) inhibited infection up to 65%, genistein (tyrosine kinase inhibitor) up to 82%, and wortmannin (PI3 kinase inhibitor) up to 63% (FIG. 3A). Next, we found that drugs that prevent actin assembly (cytochalasin D), depolymerize actin (latrunculin A), or stabilize actin filaments (jaspakinolide A) also caused dose-dependent inhibition of infection ranging between 40% and 90% (FIG. 3B). Microscopy showed that the MV-induced formation of blebs was inhibited by these actin inhibitors. An inhibitor of the Na⁺/H⁺ exchanger and macropinocytosis, EIPA, also inhibited infection by 90% (FIG. 3C). Expression of constitutive active Arf6 inhibited infection by 78% relative to cells over expressing wt Arf6 (FIG. 3D), while over-expression of dominant negative dynamin (K44A) had no impact. That infection by MV is cholesterol-dependent, and that Rac1 is required, has already been demonstrated by others (Chung et al., 2005; Locker et al., 2000). In summary, the inhibition was fully consistent with a macropinocytic process.

The macropinocytic nature of the endocytic process was also consistent with the simultaneous internalization of a fluid phase marker. The endocytic vacuoles that contained the fluorescent MV were positive for the fluid-phase marker 568-dextran, but not for 568-transferrin, a ligand internalized by clathrin-mediated endocytosis (FIG. 3E). Inclusion of 100 μM EIPA prevented internalization of MV into FITC-dextran positive vacuoles (FIG. 3E).

FIG. 3 depicts vaccinia MVs utilize macropinocytosis to enter cells. FIGS. 3A and 3B depict general kinase and actin cytoskeletal requirements of MV infection. A series of kinase inhibitors and actin-affecting drugs were assessed for their effect on MV infectivity by FACS analyses. Assays were performed as outlined in the Materials and Methods Section, below. Results are the average of three experiments presented as the percentage of infected cells relative to untreated controls. FIG. 3C depicts inhibition of MV infectivity by EIPA. Cells were pretreated with EIPA (5-N-ethyl-N-isopropyl-amiloride; Sigma Aldrich) an inhibitor of the Na⁺/H⁺ exchanger and macropinocytosis followed by infection with EGFP-MV and analyzed by FACS as per Materials and Methods Section, below. Experiments were performed in triplicate and results displayed as the percentage of infected cells relative to control infections in the absence of drug. FIG. 3D depicts effect of dominant-negative Arf6 on MV infectivity. HeLa cells were transiently transfected with fluorescent-tagged versions wild type Arf6 (WT) or the constitutive active version of Arf6 (C/A). Cells were infected with EGFP-MV at an MOI of 1. At 4 hpi cells were fixed and stained for actin. Cells were analyzed by confocal microscopy for transfected proteins (red), viral infection (green) and actin (blue). Experiments were performed in triplicate and 100 transfected cells per experiment scored for infection. Results are displayed as the average percentage of transfected/infected cells. FIG. 3E depicts internalization of MVs into endocytic vacuoles. Cells were left untreated or were pretreated with 100 μM EIPA. Cells were left uninfected or were bound with mYFP-MVs at 4° C. for 1 h at an MOI of 1. Cells were washed 2× with cold PBS and shifted to 37° C. for 15 m. Cells were then pulsed for 10 min with the fluid-phase marker 10 kDa 568-dextran (0.5 mg/ml) or 568-transferrin (Tfn) (200 ng/ml) in the presence or absence of EIPA. Surface bound dextran and Tfn was removed with a brief low pH wash prior to fixation. Samples were analyzed by confocal microscopy. Ten Z-stackes per image were collected and displayed as a Z-projection.

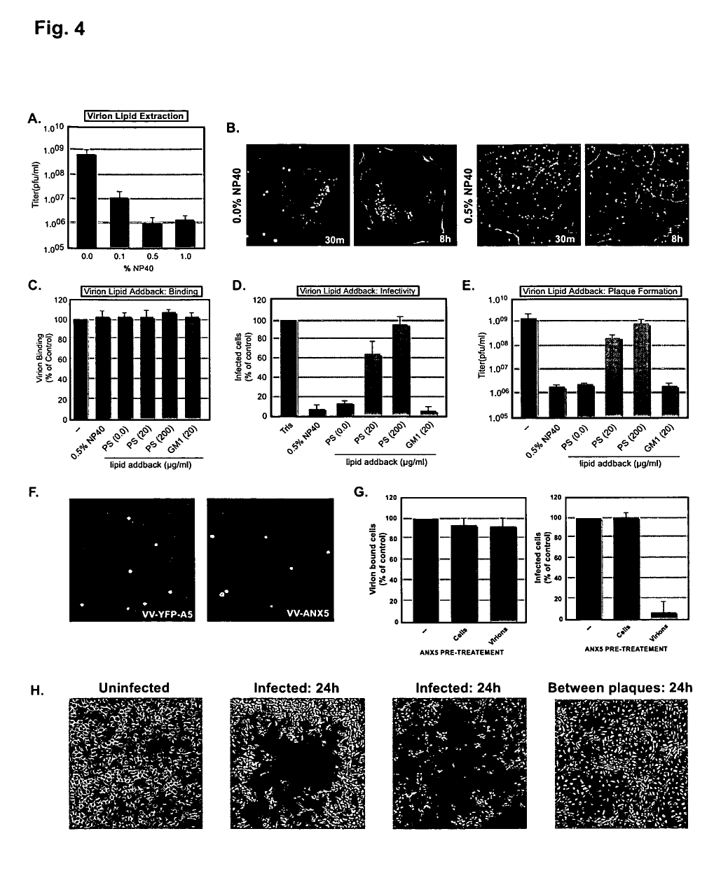

To analyze the role of phosphatidylserine (PS) in more detail, we extracted isolated MV particles using 0.5% NP40, and subsequently removed the detergent and the extracted lipids. This resulted in a dramatic drop in infectivity (FIG. 4A), but no loss of viral structural proteins (data not shown). Using fluorescent MVs, we found that the extracted viruses bound to cells but they were unable to induce blebbing and endocytosis. Even after 8 hours—when untreated MVs were found clustered inside the cells—the extracted virions were still at the cell surface suggesting a defect in the endocytosis (FIG. 4B). To test the role of PS, we restored the lipids to viruses delipidated by incubating them in the presence of liposomes with different lipid compositions. Regardless of which lipids were used, the relipidated viruses were able to bind cells (FIG. 4C). However, only those that had PS were able to induce bleb formation and endocytosis. Readdition of PS, moreover, rescued a large fraction (90%) of the original infectivity and plaque formation (FIGS. 4D and E).

A PS-binding protein, annexin-A5 (ANX5) in its EGFP tagged form was used to demonstrate that viral PS was actually exposed in the external leaflet of the viral membrane. The viruses were brightly stained when exposed to this reagent (FIG. 4F), and it was found that masking of the viral PS with ANX5 inhibited infection by 95% without affecting MV binding to cells (FIG. 4G). When cells were treated with ANX5 prior to addition of the virus, no effect on viral binding or infectivity was observed. These results showed that the PS in the virus particles was necessary to trigger the macropinocytosis process.

Finally, ANX5 was used to determine whether the lysis of infected cells that release MVs from infected cells was caused by apoptosis or necrosis. We analyzed viral plaques for the presence of surface exposed PS, a hallmark of cellular apoptosis (Martin et al., 1995). The cells surrounding viral plaques were found to contain high amounts of surface exposed PS while uninfected cells or cells between plaques did not (FIG. 4H). This indicated that in late stages of infection, vaccinia virus induces apoptosis and that this is how the MVs escape. The viruses are thus exposed to neighboring non-infected cells together with apoptotic bodies from the infected cells.

FIG. 4 depicts vaccinia MVs require PS for internalization. FIG. 4A depicts viral lipids are required for MV infectivity. Virions were subjected to lipid extraction with varying concentrations of NP40 (0.1-1.0%). After collection, virion infectivity was measured by titration (pfu/ml) on BSC40 cells. FIG. 4B depicts lipid-extracted MVs can bind but are unable to enter cells. Untreated or mYFP-MVs treated with 0.5% NP40 were added to cells at an MOI of 1. The cells were fixed at either 30 mpi or 8 hpi, stained for actin and visualized by confocal microscopy for virus binding and infection. FIG. 4C depicts binding of MVs is not dependent on lipid constituents of the virion membrane. 1×10⁹ mYFP-MVs were untreated or subjected to lipid extraction or subsequent add back with different lipids (M and M's). After add back virions were bound to HeLa cells for 1 h at 4° C., washed 2× with cold PBS and analyzed by FACS analysis as per Materials and Methods section, below. Results are the representation of three independent experiments. FIG. 4D depicts infectivity of MVs is dependent upon PS within the virion membrane. 1×10⁹ EGFP-MVs were untreated or subjected to lipid extraction and add back with different lipids (Materials and Methods section, below). After add back virions were bound to HeLa cells for 1 h at 4° C., washed 2× with cold PBS, and infection allowed to proceed for 2 h at 37° C. prior to FACS analysis. Results are the representation of three independent experiments. FIG. 4E depicts productive infection by MVs is dependent upon PS within the virion membrane. 1×10⁹ WR MVs were untreated or subjected to lipid extraction and add back with different lipids. After addback virion infectivity was measured by titration (pfu/ml) on BSC40 cells. Results are the representation of three independent experiments. FIG. 4F depicts viral membrane PS is exposed on the surface of MVs. The presence of PS on the viral membrane was demonstrated using recombinant 488 annexin V (ANX5). Virions were analyzed using an adaptation of the protocol provided in the Vybrant® Apoptosis Assay kit #2 (Molecular Probes). Briefly virions were incubated in 25 μl 1× annexin binding buffer with 2 μl 488 ANX5 at room temperature for 15 m. Virions were pelleted, washed 1× in binding buffer and bound to coverslips prior to visualization by confocal microscopy. Visualization of mYFP-MVs served as positive control. FIG. 4G depicts masking of MV membrane PS prevents infection. Binding: HeLa cells or mYFP-MVs were pretreated with ANX5 according to manufacture's (cells) or above conditions (MVs). After treatment ANX5 bound cells were incubated with untreated virions or ANX5-treated mYFP virions with untreated cells. Binding of mYFP-MVs to HeLa cells served as a positive control. Experiments were done in triplicate and results displayed as the % of virion bound cells relative to controls. Infection: Experiments were performed as with binding using EGFP-MVs. FIG. 4H depicts viral plaques are enriched for apoptotic cells. BSC40 cell monolayers infected with wt-MVs and infection allowed to proceed for 24 h. Cells were then washed 2× in PBS and analyzed for live, apoptotic and necrotic cells as per manufacture's protocol (Vybrant® Apoptosis Assay kit #2; Molecular Probes). 488 ANX5 is shown in green and propidium iodide in red. Uninfected cells were utilized for control staining.

### Materials and Methods:

### Cell Lines and Viruses

Monolayers of BSC40 primate and HeLa ATCC cells were maintained in Dulbecco modified Eagle medium (DMEM; Gibco BRL) containing 10% fetal calf serum (FCS) at 37° C. Wild-type (wt) vaccinia virus (strain WR), WR E/L EGFP (MV-EGFP) (kindly provided by Paula Traktman, Medical College of Wisconsin), and WR containing a fluorescent version of the core protein, A5 (mYFP-MV), were used as indicated. All viral stocks were prepared in the presence of Brefeldin A. Virus was purified from cytoplasmic lysates by ultracentrifugation through 36% sucrose banding on 25 to 40% sucrose gradients.

### Fluorescence Activated Cell Sorting (FACS)

Binding assay: mYFP-MV was allowed to bind to HeLa cells (wt or treated) at 4° C. in serum free DMEM for 1 h at a multiplicity of infection (MOI) of 1. Virion-bound cells were shifted to 4° C., washed 2× in PBS, trypsinized from the plate and fixed in formaldehyde (FA) for 30 m on ice. Fixed cells were collected by centrifugation and washed 1× in PBS, recollected and suspended in PBS for FACS analysis. A total of 10,000 events were analyzed from each sample and scored for mYFP expression relative to unbound and mYFP-MV bound controls. Infection assay: EGFP-MV was allowed to bind to HeLa cells at 4° C. in serum free DMEM in the presence of drug for 1 h. All assays were performed at a MOI of 1. Post-binding, cells were washed 2 times with cold PBS followed by the addition of pre-warmed media. Cells were shifted to 37° C. and infection allowed to proceed for 2 h prior to fixation and preparation for FACS as above. Drug screening: HeLa cells were pretreated with the indicated drug at varying concentrations for 15 m prior to infection. Cells were bound with EGFP-MV at 4° C. in serum free DMEM in the presence of drug for 1 h. All assays were performed at an MOI of 1. Post-binding, cells were washed 2 times with cold PBS followed by the addition of pre-warmed media containing drug. Cells were then shifted to 37° C. and infection allowed to proceed for 2 h. Cells were fixed and prepared as above for FACS analysis. All FACS analyses were displayed as the percentage of infected cells relative to control infections in the absence of drug.

### Virion Detergent Extraction

A total of 1.0×10⁹ vaccinia viruses wt, EGFP-MV, or mYFP-MV virions (purified as described above) were incubated at 37° C. for 60 min in a reaction mixture containing 100 mM Tris (pH 9.0) and various concentrations of NP-40: 0, 0.1, 0.5, and 1.0% (vol/vol). After the extraction solubilized and particulate fractions representing the membrane and core components of the virion, respectively, were separated by sedimentation (16,000×g, 30 min, room temperature). Samples were then subjected to titration on BSC40 cells to determine the viral titer (number of PFU/milliliter). All titers were performed in triplicate, and the results were averaged.

### Virion Lipid Addbacks

Liposomes with different lipid composition were prepared and lipid extracted virions reconstituted according to the methods of Oie (Oie, 1985 #2008). Briefly, lipid extracted virions were incubated with PC based liposomes (200 μg/ml) incorporated with varying concentrations of PS (20 μg/ml or 200 μg/ml) or GM1 (20 μg/ml) at 37° C. for 2 h. Virions were collected by centrifugation, subsequently washed, and resuspended in buffer. Reconstituted virions were subject to FACS and microscopy based binding and entry assays as well as tittering for viral yield.

### Example Two

### Evaluation of Entry Mechanism of Adenovirus Serotype 3 Virus Materials & Methods

### Cells and Viruses

Cells were grown in DME (GIBCO-BRL) containing 10% FCS (GIBCO-BRL) at low passage number as described (Suomalainen et al., 1999). Human melanoma M21 litter (negative for surface-expressed av integrins) and M21L cells (positive for cell surface αv integrins) were from Dr. D. Cheresh (Scripps Research Institute, La Jolla, Calif., Felding-Habermann et al., 1992). K562 chronic myelogenous leukemia cells were grown as described (Nagel et al., 2003). BHK cells stably expressing CD46 or CAR were produced by stably transfecting plasmids encoding either for the BC1 isoform of CD46 or CAR (Sirena et al., 2005). Ad3 and Ad2 ts1 were grown and isolated as described (Greber et al., 1996). Labeling of Ad3 with texas red was as published (Nakano and Greber, 2000). (3H)-thymidine-labeled Ad3 was produced as published (Greber et al., 1993).

cDNAs, Proteins and Chemicals

cDNAs encoding CtBP1-S/BARS were obtained from Dr. A. Colanzi (Dep. Of Cell Biology and Oncology, S. Maria Imbaro, Italy). pCMV-myc CtBP1-S wt was generated by ligation of the PCR amplified CtBP1-S wt (digested with Sal I and Not I, respectively) into the pCMV backbone vector (Stratagene). Myc-CtBP3 D355A was generated with the QuikChangeR site-directed mutagenesis kit (Stratagene) with the primers 5′-CTGGGCCAGCATGGCCCCTGCTGTGGTG-3′ (SEQ ID NO: 21) and 5′-CACCACAGCAGGGGCCATG CTGGCCCAG-3′ (SEQ ID NO: 22) (Bonazzi et al., 2005). The obtained cDNA was verified by sequencing. K44A-dyn2 and dyn2 wt expression plasmid were from Dr. C. Lamaze (Pasteur Institute). Pak1 wt and inhibitory domain expression vectors were from J. Chernoff (Fox Chase Cancer Center, Philadelphia, Pa.). Toxin B (0.5 mg/ml) was from Drs. F. Hofmann and K. Aktories (University of Freiburg, Freiburg, Germany). The PKC inhibitors Go 6976 (1 μM) and Go 6983 (1 μM) were purchased from Calbiochem (Juro Supply), the Na+/H+ exchanger inhibitor EIPA (100 μM) was from Alexis Corporation, Cytochalasin D (5 μM) and Jasplakinolide (500 nM) from Calbiochem. Cholesterol depletion by methyl-beta-cyclodextrin (50 mM) was performed as published earlier (Imelli et al., 2004). Ad3 soluble fiber knob (used at a final concentration of 5 μg/ml) was from P. Fender (Grenoble, France). Dynasore was kindly synthesized by Dr. J. S. Siegel (Organic Chemistry Institute, University of Zurich). Antibody against CtBP1-L/S were from BD Transduction laboratories, PAK1 antibody (C-19) was from Santa Cruz Biotechnology (Santa Cruz, Calif.), and antibody against phosphorylated PAK1 (T423) was from Cell Signaling Technology.

### Endocytosis and Ad-eGFP Transductions

Cells were incubated with (3H)-thymidine-labeled Ad3 (1 μg/ml; 3×10⁹ particles on a 35 mm dish) in the cold for 60 min in RPMI-0.2% BSA, washed with cold RPMI-BSA and warmed with RPMI-BSA for the indicated time points. Cells were washed twice with cold RPMI-BSA and cold PBS. To remove the extracellular virus particles from the cells, cells were incubated with cold 2% trypsin-EDTA (GIBCO) for 60 min in the cold, PBS-2% FCS was added and the cells centrifuged at 500×g. The wash step was repeated twice. Cell lysates were prepared in hot SDS (0.4%), sheared in a 20G clinical syringe, and radioactivity was determined by fluid scintillation counting (Ready Safe; Beckman Coulter) with a Beckman Coulter Scintillation System LS 3801. Counts of control cells without trypsin were used as 100% control. For drug experiments cells were preincubated with drug in RPMI-BSA at 37° C. for 30 min. For transduction experiments cells were washed with warm RPMI-BSA and incubated with 1 μg/ml of virus for 60 min, washed several times with RPMI-BSA and incubated in a water bath for 4 h (A549 cells), 5 h (HeLa cells), 8 h (M21 and M21L cells) and 16 h (K562 cells). The cells were washed with cold PBS and treated with 2% trypsin in the cold, followed by 2% PBS-FCS and analysis by flow cytometry (Beckman FC500 cytometer). At least 10000 viable cells were counted per sample. For drug experiments cells were pretreated with inhibitors in RPMI-BSA at 37° C. for 30 min, followed by warm infection for 60 min in presence of drugs followed by washing in medium without drug and further incubation.

### Dextran Uptake

Cells were preincubated with 5 μg/ml Ad3 in the cold, washed with warm RPMI-BSA, and warmed in RPMI-BSA containing 0.5 mg/ml dextran-FITC (lysine-fixable 10 kDa, Molecular Probes) at 37° C. for 10 min, as described earlier (Meier et al., 2002). Dextran uptake was stopped by washing cells with cold RPMI-BSA and PBS (3 repeats). Surface-bound dextran was removed by acid treatment in cold 0.1 M sodium acetate pH 5.5, 0.05 M NaCl for 10 min. For FACS analysis, cells were detached with 2% trypsin in PBS (GIBCO-BRL) on ice for 25 min, transferred into 6 ml polypropylene tubes (no 2063; Falcon, Becton Dickinson) containing 2 ml of 7% FCS/PBS, pelleted at 290×g and resuspended in 2% FCS/PBS. At least 10000 viable cells were counted per sample in a flow cytometer (Beckman FC500 cytometer).

### Transmission Electron Microscopy, and Uptake of BSA-Gold

After cold binding of Ad3 or Ad2 ts1 (50 μg/ml, multiplicity of infection [MOI] of 5000) for 60 min, washing and internalization as appropriate, cells were fixed in 2% formaldehyde-1.5% glutaraldehyde in 0.1 M sodium cacodylate buffer, pH 7.4 (CaCo) overnight, and washed several times in CaCo, followed by postfixation in 1% OsO₄ (Electron Microscopa Sciences) and 1.5% potassium ferricyanide (FeK₃N₆) in double distilled water at 4° C. for 60 min (modified according to the method of Simionescu and Siminonescu). Specimens were rinsed in 0.1 M sodium cacodylate, contrasted with 1% tannic acid in 0.05 M sodium cacodylate at room temperature for 45 min, washed in 1% sodium sulfate, rinsed in H₂O, stained in 2% uranylacetate in H₂O overnight, and embedded in Epon as described previously (Nakano et al., 2000). Virus particles were quantified at 50000× magnification in ultrathin sections at the plasma membrane, endosomes and cytosol, and viewed in a transmission electron microscope (Zeiss EM 902A) at an acceleration voltage of 80,000 V. 15 nm collodial gold was prepared by citrate reduction of HAuCl₄ (Frens, 1973; Horisberger and Rosset, 1977). To 20 ml of collodial gold solution (pH adjusted to 5.9) 50 μl of 10 mg/ml BSA (Sigma, fatty acid free) solution was added (De Roe et al., 1987). To stabilize the BSA-gold complex, 1 ml of 1% PEG 20000 (Roth, Switzerland) were added, the sample centrifuged at 28′000 g for 60 min, and the pellet dissolved in 2 ml gold-buffer (sterile filtered PBS containing 0.2% PEG-20000) and stored at 4° C. BSA-gold internalization was performed after cold binding of Ad3 or Ad2-ts 1 using a 1:1 dilution of BSA-gold with RPMI-BSA (approximately 0.1 mg/ml of BSA) at 37° C. for 10 min.

### Fluorescence Microscopy and Immunofluorescence

Cells were transfected with different DNA constructs 30 h prior to experiment using Fugene 6 (Roche, according to manufacturer's instruction). Cells were infected with Ad3-eGFP or Ad5-eGFP at 37° C. for 60 min, washed and incubated at 37° C. for 15 h. Cells were fixed and mounted with DAKO. For dextran and transferrin uptake, cells were synchronized with 5 μg/ml of Ad3 in the cold, washed warm and pulsed with a mixture of 0.5 mg/ml dextran-TR and 20 μg/ml of transferrin-Alexa647 in RPMI-BSA at 37° C. for 30 min (waterbath), followed by a 5 min chase, fixed and mounted with DAKO. Confocal laser scanning microscopy (CLSM) was performed on a Leica-DM SP2 RXA2-TCS-AOBS microscope (Leica Microsystems, Wetzlar, Germany) equipped with an Ar-ArKr laser, a He—Ne 543-594 laser, a He—Ne 633 laser, a diode laser at 405 nm, and a 63× oil immersion objective (N.A. 1.4 PL APO). The pinhole value was 1.0, airy 1, yielding optical sections of ˜0.48 μm with a voxel of 0.233 by 0.233 by 0.48 μm. The zoom factor was 2. Image processing was performed with Leica and Photoshop software (Adobe), and fluorescence intensities determined using Image J (webpage: http://rsb.info.nih.gov/ij/) on cell total projections. For CtBP1-L/S colocalization with dextran-positive endosomes cells were cold synchronized with Ad3-TR (2 μg/ml) for 60 min on ice, washed with warm RPMI-BSA and pulsed with 0.5 mg/ml of dextran-FITC at 37° C. for 10 min Cells were washed extensively with RPMI-BSA and PBS, fixed and analyzed by immunofluorescence using a CtBP1-L/S mouse monoclonal antibody (BD Transduction laboratories) and a secondary Alexa647-conjugated goat anti-mouse antibody.

siRNA Transfections

K562 cells were transfected with siRNA directed against clathrin heavy chain (AACCUGCGGUCUGGAGUCAAC (SEQ ID NO: 23); Qiagen (Hinrichsen et al., 2003)) and against CtBP1/CtBP3 (CCGUCAAGCAGAUGAGACAUU (SEQ ID NO: 24); GGAUAGAGACCACGCCAGUUU (SEQ ID NO: 25); Dharmacon (Bonazzi et al., 2005)) using Nucleofector I (Amaxa; program T-03) according to the manufacturer's instructions. Transfection of non-targeting siRNA sequences (Qiagen, or Dharmacon) were used as controls. Transfections were done at day 0 and day 2, cell lysates for Western blotting and experiments were collected at day 4. HeLa cells were transfected with siRNA directed against clathrin heavy chain, CtBP1/CtBP3 or PAK1 (validated siRNA Cat. SI00605703 and SI00605696; Qiagen) using Lipofectamine 2000 (Invitrogen) according to the manufacturer's instructions. Transfections were done twice at day 0 and day 2, cell lysates for Western blotting and experiments collected at day 4. A549 cells were transfected with siRNA directed against clathrin heavy chain, CtBP1/CtBP3, PAK1 or dynamin2 {GACAUGAUCCUGCAGUUCA (SEQ ID NO: 26), Qiagen, \Huang, 2004 #15509} using Lipofectamine 2000 as described above.

### Western Blotting

Cells were grown in 35-mm dishes, washed with phosphate-buffered saline (PBS) and lysed in 500 μl of 2% hot SDS. The lysate was passed through a 20-gauge needle several times and heated to 95° C. for 30 s. After centrifugation at 16,000×g for 10 min, 150 μl of the supernatant was mixed with 50 μl of sample buffer (200 mM Tris/HCl, pH 6.8, 8% SDS, 0.4% bromphenol blue, 40% glycerol, 167 mM dithiothreitol) and heated to 95° C. for 10 min. Extracts were separated on 10% SDS-PAGE, transferred to Hybond-ECL nitrocellulose membrane (Amersham Biosciences, Zurich, Switzerland), and blocked with 5% dried milk in 50 mM Tris/100 mM sodium chloride/0.1% Tween, pH 7.4 (TNT). After immunological probing (with 3% milk for the PAK1 blots, with 0.2% BSA for the CtBP1 blots) HRP-conjugated antibodies were detected with ECL Plus reagents (Amersham Biosciences). Filters were stripped with 100 mM μ-mercaptoethanol, 2% SDS, 62.5 mM Tris/HCl, pH 6.7, at 50° C. for 30 min, washed extensively with TNT, blocked with 5% dried milk, and reprobed with an anti-calnexin antibody (a kind gift of Dr. A. Helenius, Zürich) in TNT with 3% milk.

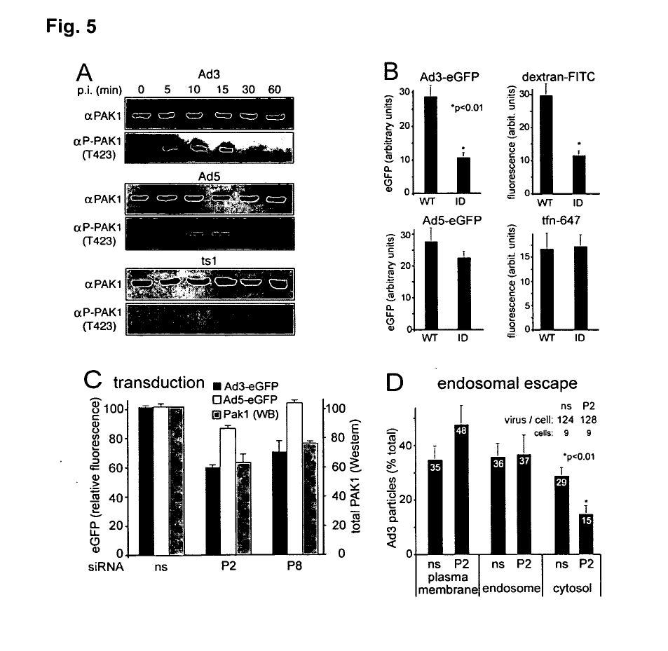

FIG. 5 depicts activation of PAK1 required for Ad3 but not Ad5 endocytosis and infection. FIG. 5A depicts Ad3 and Ad5 activate PAK1. HeLa cells were incubated with 0.5 μg/ml of Ad3, Ad5 or ts1 (approx 5×10⁵ cells) in the cold for 60 min, washed and warmed for different times. Cells were washed with cold PBS containing phosphatase and protease inhibitors, scraped off the dish, resuspended in 100 μl of PBS with inhibitors, mixed with SDS sample buffer containing dithiothreitol, heated and analyzed by SDS-PAGE (10⁵ cell equivalents per lane), and Western blotting against PAK1 and phosphorylated PAK1 (T423), respectively, using ECL as a detection method. FIG. 5B: HeLa cells expressing wild type or dominant negative PAK1 (inhibitory domain ID) were transduced with Ad3-eGFP or Ad5-eGFP, or assessed for uptake of dextran-FITC or transferrin labeled with Alexa 647 upon Ad3 infection. FIG. 5C: HeLa cells were transfected with siRNAs P2 and P8 against PAK1 or ns siRNA for 72 h (double transfection, 20 pmoles/ml siRNA), infected with Ad3-eGFP or Ad5-eGFP for 6 h, and analyzed for eGFP expression by flow cytometry. 1×10 exp5 of transfected cells were analyzed by Western blotting (WB) for PAK1 contents. FIG. 5D: Endosomal escape of Ad3 was measured by thin section EM in HeLa cells transfected with anti-PAK1 siRNA P2 and ns siRNA, respectively. Results show virions at the plasma membrane, in endosomes and the cytosol.

### Example Three

### Role of EGFR During Viral Infection

The Rac1 effector, PAK1, is required for the entry of vaccinia mature virions (MVs), and the RhoA family GTPase Rac1 and RhoA are activated during the entry process (Mercer and Helenius (2008) Science 320:531-535). In turn, these GTPases can be activated by a variety of cell surface receptors (Schiller (2006) Cell Signal 18:1834-1843). Amongst these is the epidermal growth factor receptor (EGFR, Erb1). The involvement of the EGFR in vaccinia entry has been controversial (Marsh and Eppstein (1987) J Cell Biochem. 34:239-245; Eppstein et al. (1985) Nature 318:663-665; Hugin and Hauser (1994) J. Virol. 68:8409-8412).

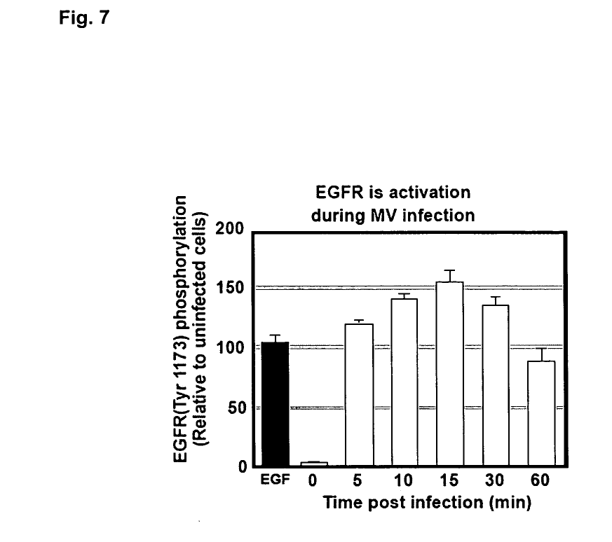

An MV infection timecourse was used to assess the activation status of EGFR during vaccinia infection. EGFR activation was monitored using an antibody that recognizes EGFR phosphorylated at tyrosine 1173. EGFR was robustly activated within five minutes of virus addition, peaking at 15 minutes post infection (FIG. 7).

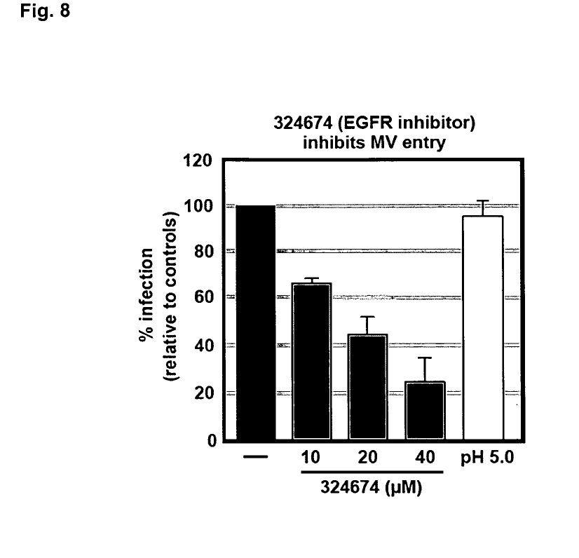

The EGFR inhibitor 324674 (Calbiochem) effectively blocks MV entry, and is readily by-passed by low-pH fusion (FIG. 8)

These results indicate that vaccinia MVs can activate EGFR during infection, and that this activation is required for entry. In addition they suggest that MV induced activation of PAK1 lies downstream of the EGFR. These data provide additional insight into the signaling pathways required for MV entry and infection and suggest other potential cellular antiviral targets and theraputics against poxvirus infection.

### Materials and Methods:

Fluorescence Activated Cell Sorting (FACS) Infection assay.

Drug screening: HeLa cells were pretreated with the indicated drugs at varying concentrations for 15 min prior to infection. EGFP-EXPRESS-MV was allowed to bind at 4° C. in serum free DMEM in the presence of drug for 1 h. All assays were performed at a MOI of 1. After binding, cells were washed twice with cold PBS followed by the addition of pre-warmed media containing drug. Cells were shifted to 37° C. and infection was allowed to proceed for 2 h. The cells were washed twice in PBS, trypsinized from the plate, and fixed in 4% formaldehyde (FA) for 30 min on ice. The fixed cells were collected by centrifugation, washed in PBS, recollected, and suspended in PBS for FACS analysis using a FACSCalibur System (BD Biosciences). All FACS analyses were performed in triplicate and displayed as the average percentage of infected cells relative to control infections in the absence of drug. Error bars represent the standard deviation between experiments.

### REFERENCES

Bangham, A. D., M. M. Standish, and J. C. Watkins 1965. Diffusion of univalent ions across the lamellae of swollen phospholipids. J. Mol. Biol. 13(1): 238-52.
Birault, V., C. J. Harris, J. Le, M. Lipkin, R. Nerella, and A. Stevens. 2006. Bringing kinases into focus: efficient drug design through the use of chemogenomic toolkits. Curr. Med. Chem. 13(15): 1735-48,
Bui M, Whittaker G, Helenius A (1996) The protein kinase inhibitor, H7, directly blocks Mediated nuclear export of influenza virus RNPs. submitted.
Bukrinskaya A G, Vorkunova N K, Kornilayeva G V, Naramanbetova R A, Vorkunova G K (1982) Influenza virus uncoating in infected cells and effect of rimantadine. J Gen Virol 60: 49-59.
Cros J F, Garcia-Sastre A, Palese P (2005) An unconventional NLS is critical for the nuclear import of the influenza A virus nucleoprotein and ribonucleoprotein. Traffic 6(3): 205-213.
Doms R W, Lamb R A, Rose J K, Helenius A (1993) Folding and assembly of viral membrane proteins. Virology 193(2): 545-562.
Ehrhardt C, Marjuki H, Wolff T, Nurnberg B, Planz O et al. (2006) Bivalent role of the phosphatidylinositol-3-kinase (PI3K) during influenza virus infection and host cell defense. Cell Microbiol 8(8): 1336-1348.
Graeser R U, Sarah; Brosig, Michaela; Kubbutat, Michael H. G. (2004) Highly efficient gene silencing using 2-for-Silencing siRNA Duplexes targeting protein kinase genes. QIAGEN News 2004 e32.
Huang Y T, Turchek B M (2000) Mink lung cells and mixed mink lung and A549 cells for rapid detection of influenza virus and other respiratory viruses. J Clin Microbiol 38(1): 422-423.
Khor R, McElroy L J, Whittaker G R (2003) The ubiquitin-vacuolar protein sorting system is selectively required during entry of influenza virus into host cells. Traffic 4(12): 857-868.
Kielian M, Rey F A (2006) Virus membrane-fusion proteins: more than one way to make a hairpin. Nat Rev Microbiol 4(1): 67-76.
Ludwig S (2006) Signaling and apoptosis in influenza virus-infected cells. In: Kawaoka Y, editor. Influenza Virology: Current Topics by Yoshihiro Kawaoka: Caister Academic Press, pp. 37-64.
Maeda T, Kawasaki K, Ohnishi S (1981) Interaction of influenza virus hemagglutinin with target membrane lipids is a key step in virus-induced hemolysis and fusion a pH 5.2. Proc Natl Acad Sci USA 78: 4133-4137.
Manning G, Whyte D B, Martinez R, Hunter T, Sudarsanam S. (2002) The protein kinase complement of the human genome. Science 298(5600):1912-34.
Marsh M, Helenius A (1989) Virus entry into animal cells. Adv Virus Res 36: 107-151.
Martin K, Helenius A (1991) Nuclear transport of influenza virus ribonucleoproteins: the viral matrix protein (M1) promotes export and inhibits import. Cell 67: 117-130.
Matlin K S, H. Reggio, A. Helenius, and K. Simons (1982) Infectious entry pathway of influenza virus in a canine kidney cell line. J Cell Biol 91: 601-613.
Matrosovich M, Matrosovich T, Garten W, Klenk H D (2006) New low-viscosity overlay medium for viral plaque assays. Virol J 3(1): 63.
Matrosovich M, N.; Klenk, Hans-Dieter; Kawaoka, Yoshihiro (2006) Receptor specificity, host range, and pathogenicity of influenza viruses. In: Kawaoka Y, editor. Influenza Virology: Current Topics by Yoshihiro Kawaoka Caister Academic Press, pp. 37-64.
Matrosovich M N, Matrosovich T Y, Gray T, Roberts N A, Klenk H D (2004a) Human and avian influenza viruses target different cell types in cultures of human airway epithelium. Proc Natl Acad Sci USA-101(13): 4620-4624.
Matrosovich M N, Matrosovich T Y, Gray T, Roberts N A, Klenk H D (2004b) Neuraminidase is important for the initiation of influenza virus infection in human airway epithelium. J Virol 78(22): 12665-12667.
O'Neill R E, Jaskunas R, Blobel G, Palese P, Moroianu J (1995) Nuclear import of influenza virus RNA can be mediated by viral nucleoprotein and transport factors required for protein import. J Biol Chem 270: 22701-22704.
Pleshka S, Ludwig S, Wolff T, Planz (2006) Anti-viral approaches against infleunza viruses. In: Bogner E, Holzenburg A, editors. Newconceptys of antiviral therapy. Dordrecht: Springer, pp. 115-188.
Rodriguez-Boulan E (1983) Polarized assembly of enveloped viruses from cultured epithelial cells. Methods in Enzymology 98: 487-501.
Rust M J, Lakadamyali M, Zhang F, Zhuang X (2004) Assembly of endocytic machinery around individual influenza viruses during viral entry. Nat Struct Mol Biol 11(6): 567-573.
Sieczkarski S B, Whittaker G R (2002) Influenza virus can enter and infect cells in the absence of clathrin-mediated endocytosis. J Virol 76(20): 10455-10464.
Sieczkarski S B, Brown H A, Whittaker G R (2003) Role of protein kinase C betall in influenza virus entry via late endosomes. J Virol 77(1): 460-469.
Skehel J, Wiley D (2000) Receptor binding and membrane fusion in virus entry: the influenza hemagglutinin. Annu Rev Biochem 69: 531-569.
Szoka, F. Jr., and D. Papahadjopoulos. 1978. Procedure for preparation of liposomes with large internal aqueous space and high capture by reverse-phase evaporation. Proc. Natl. Acad. Sci. USA. 75(9): 4194-8.
White J, Kartenbeck J, Helenius A (1982) Membrane fusion activity of Influenza virus. EMBO J. 1:217-222.
Whittaker G R D, P. (2006) Entry and intracellular transport of influenza virus. In: Kawaoka Y, editor. Influenza Virology: Current Topics by Yoshihiro Kawaoka: Caister Academic Press, pp. 37-64.
Wurzer W J, Planz O, Ehrhardt C, Giner M, Silberzahn T et al. (2003) Caspase 3 activation is essential for efficient influenza virus propagation. Embo J 22(11): 2717-2728.
Yoshimura A, Ohnishi S (1984) Uncoating of influenza virus in endosomes. Journal of Virology 51 (2): 497-504.
Bonazzi, M., S. Spano, G. Turacchio, C. Cericola, C. Valente, A. Colanzi, H. S. Kweon, V. W. Hsu, E. V. Polishchuck, R. S. Polishchuck, M. Sallese, T. Pulvirenti, D. Corda, and A. Luini. 2005. CtBP3/BARS drives membrane fission in dynamin-independent transport pathways. Nat Cell Biol. 7:570-80.
Charras, G. T., C. K. Hu, M. Coughlin, and T. J. Mitchison. 2006. Reassembly of contractile actin cortex in cell blebs. J. Cell Biol. 175:477-90.
Chung, C. S., C. Y. Huang, and W. Chang. 2005. Vaccinia virus penetration requires cholesterol and results in specific viral envelope proteins associated with lipid rafts. J. Virol. 79:1623-34.
Damke, H., T. Baba, D. E. Warnock, and S. L. Schmid. 1994. Induction of mutant dynamin specifically blocks endocytic coated vesicle formation. J. Cell Biol. 127:915-34.
De Roe, C., P. J. Courtoy, and P. Baudhuin. 1987. A model of protein-colloidal gold interactions. J Histochem Cytochem. 35:1191-8.
Dharmawardhane, S., A. Schurrnann, M. A. Sells, J. Chernoff, S. L. Schmid, and G. M. Bokoch. 2000. Regulation of macropinocytosis by p21-activated kinase-1. Mol Biol Cell. 11:3341-52.
Falcone, S., E. Cocucci, P. Podini, T. Kirchhausen, E. Clementi, and J. Meldolesi. 2006. Macropinocytosis: regulated coordination of endocytic and exocytic membrane traffic events. J Cell Sci. 119:4758-69.
Felding-Habermann, B., B. M. Mueller, C. A. Romerdahl, and D. A. Cheresh. 1992. Involvement of integrin alpha V gene expression in human melanoma tumorigenicity. J Clin Invest. 89:2018-22.
Fishkind, D. J., L. G. Cao, and Y. L. Wang. 1991. Microinjection of the catalytic fragment of myosin light chain kinase into dividing cells: effects on mitosis and cytokinesis. J Cell Biol. 114:967-75.
Frens, G. 1973. Controlled nucleation for the regulation of particle size in monodisperse gold suspensions. Nature Phys Sci. 241:20-12.
Greber, U. F., P. Webster, J. Weber, and A. Helenius. 1996. The role of the adenovirus protease in virus entry into cells. EMBO J. 15:1766-1777.
Greber, U. F., M. Willetts, P. Webster, and A. Helenius. 1993. Stepwise dismantling of adenovirus 2 during entry into cells. Cell. 75:477-486.
Grimmer, S., B. van Deurs, and K. Sandvig. 2002. Membrane ruffling and macropinocytosis in A431 cells require cholesterol. J Cell Sci. 115:2953-62.
Hewlett, L. J., A. R. Prescott, and C. Watts. 1994. The coated pit and macropinocytic pathways serve distinct endosome populations. J Cell Biol. 124:689-703.
Hinrichsen, L., J. Harborth, L. Andrees, K. Weber, and E. J. Ungewickell. 2003. Effect of clathrin heavy chain- and alpha-adaptin-specific small inhibitory RNAs on endocytic accessory proteins and receptor trafficking in HeLa cells. J Biol. Chem. 278:45160-70.
Horisberger, M., and J. Rosset. 1977. Colloidal gold, a useful marker for transmission and scanning electron microscopy. J Histochem Cytochem. 25:295-305.
Imelli, N., O. Meier, K. Boucke, S. Hemmi, and U. F. Greber. 2004. Cholesterol is required for endocytosis and endosomal escape of adenovirus type 2. J Virol. 78:3089-98.
Kirkham, M., and R. G. Parton. 2005. Clathrin-independent endocytosis: new insights into caveolae and non-caveolar lipid raft carriers. Biochim Biophys Acta. 1746:349-63.
Limouze, J., A. F. Straight, T. Mitchison, and J. R. Sellers. 2004. Specificity of blebbistatin, an inhibitor of myosin II. Muscle Res Cell Motil. 25:337-41.
Locker, J. K., A. Kuehn, S. Schleich, G. Rutter, H. Hohenberg, R. Wepf, and G. Griffiths. 2000. Entry of the two infectious forms of vaccinia virus at the plasma membane is signaling-dependent for the IMV but not the EEV. Mol. Biol. Cell. 11:2497-2511.
Martin, S. J., C. P. Reutelingsperger, A. J. McGahon, J. A. Rader, R. C. van Schie, D. M. LaFace, and D. R. Green. 1995. Early redistribution of plasma membrane phosphatidylserine is a general feature of apoptosis regardless of the initiating stimulus: inhibition by overexpression of Bc1-2 and Abl. J Exp Med. 182:1545-56.
Meier, O., K. Boucke, S. Vig, S. Keller, R. P. Stidwill, S. Hemmi, and U. F. Greber. 2002. Adenovirus triggers macropinocytosis and endosomal leakage together with its clathrin mediated uptake. J. Cell Biol. 158:1119-1131.
Mills, J. C., N. L. Stone, J. Erhardt, and R. N. Pittman. 1998. Apoptotic membrane blebbing is regulated by myosin light chain phosphorylation. J. Cell Biol. 140:627-36.
Nagel, H., S. Maag, A. Tassis, F. O, Nestle, U. F. Greber, and S. Hemmi. 2003. The alphavbeta5 integrin of hematopoietic and nonhematopoietic cells is a transduction receptor of RGD-4C fiber-modified adenoviruses. Gene Ther. 10:1643-53.
Nakano, M. Y., K. Boucke, M. Suomalainen, R. P. Stidwill, and U. F. Greber. 2000. The first step of adenovirus type 2 disassembly occurs at the cell surface, independently of endocytosis and escape to the cytosol. J. Virol. 74:7085-95.
Nakano, M. Y., and U. F. Greber. 2000. Quantitative microscopy of fluorescent adenovirus entry. J. Struct. Biol. 129:57-68.
Sirena, D., Z. Ruzsics, W. Schaffner, U. F. Greber, and S. Hemmi. 2005. The nucleotide sequence and a first generation gene transfer vector of species B human adenovirus serotype 3. Virology. 343:283-98.
Suomalainen, M., M. Y. Nakano, K. Boucke, S. Keller, R. P. Stidwill, and U. F. Greber. 1999. Microtubule-dependent minus and plus end-directed motilities are competing processes for nuclear targeting of adenovirus. J. Cell Biol. 144:657-672.
Townsley, A. C., A. S. Weisberg, T. R. Wagenaar, and B. Moss. 2006. Vaccinia virus entry into cells via a low-pH-dependent endosomal pathway. J. Virol. 80:8899-908.
Veithen, A., P. Cupers, P. Baudhuin, and P. J. Courtoy. 1996. v-Src induces constitutive macropinocytosis in rat fibroblasts. J Cell Sci. 109 (Pt 8):2005-12.

What is claimed is:

1. A method of treating a poxvirus infection comprising administering to an animal subject in need thereof an effective amount of a kinase modulator.

2. The method of claim 1 wherein the animal subject is a human.

3. The method of claim 2 wherein the kinase modulator modulates a kinase selected from the group consisting of PAK1; DYRK3; PTK9; and GPRK2L.

4. The method of claim 1 wherein said kinase modulator is a host cell kinase modulator.

5. The method of claim 1 wherein the kinase modulator is a dominant negative molecule targeting the kinase, an siRNA, an shRNA an antibody or a small molecule.

6. The method of claim 1 wherein the kinase modulator is an siRNA.

7. The method of claim 4 wherein said host cell kinase modulator is a host cell kinase inhibitor.

8. The method of claim 7 wherein said host cell kinase inhibitor is an inhibitor of a kinase selected from the group consisting of PAK1; DYRK3; PTK9; and GPRK2L.

9. The method of claim 1 where in the kinase modulator is CEP-1347.

10. The method of claim 1 wherein the poxvirus is a variola virus.

11. The method of claim 1 wherein the poxvirus is a vaccinia virus

12. The method of claim 1 wherein the infection is a respiratory infection.

13. A method of treating a virus infection comprising administering to an animal subject in need thereof an effective amount of a modulator of a macropinocytosis pathway.

14. The method of claim 13 wherein the animal subject is a human.

15. The method of claim 13 wherein said modulator of a macropinocytosis pathway is an inhibitor of said macropinocytosis pathway.

16. The method of claim 15 wherein said inhibitor is a kinase inhibitor.

17. The method of claim 15 wherein said inhibitor is a host cell kinase inhibitor.

18. The method of claim 17 wherein said host cell kinase inhibitor is an inhibitor of a kinase selected from the group consisting of PAK1; DYRK3; PTK9; and GPRK2L.

19. The method of claim 18, wherein the inhibitor is CEP-1347.

20. The method of claim 13 wherein said virus is a pox virus.

21. The method of claim 13 wherein said virus is a variola virus.

22. The method of claim 13 wherein said virus is a vaccinia virus.

23. A method comprising:
a) contacting a cell with a kinase inhibitor and virus and
b) determining whether the kinase inhibitor inhibits infection of the cell by the virus.

24. The method of claim 23 wherein the virus is a pox virus

25. The method of claim 23 wherein the virus is an influenza virus.

26. The method of claim 23 wherein the virus is vaccinia or variola.

27. The method of claim 23 wherein the kinase inhibitor inhibits a kinase selected from the group consisting of PAK1; DYRK3; PTK9; and GPRK2L.

28. The method of claim 23 wherein the kinase inhibitor is selected from the group consisting of dominant negative molecule targeting the kinase, an siRNA, an shRNA an antibody or a small molecule

29. The method of claim 23 wherein the contacting is performed in vitro.


SEQUENCE LISTING (49 sequences)

SEQ ID NO: 1 - protein, 74 aa, Homo sapiens
MSNNGLDIQDKPPAPPMRNTSTMIGAGSKDAGTLNHGSKPLPPNPEEKKKKDRFYRSILPGDKTNKKKEKERPE

SEQ ID NO: 2 - protein, 13 aa, Homo sapiens
KPPAPPMRNTSTM

SEQ ID NO: 3 - protein, 25 aa, Human immunodeficiency virus
YGRKKRRQRRRGKPPAPPMRNTSTM

SEQ ID NO: 4 - protein, 67 aa, Homo sapiens
HTIHVGFDAVTGEFTGMPEQWARLLQTSNITKSEQKKNPQAVLDVLEFYNSKKTSNSQKYMSFTDKS

SEQ ID NO: 5 - DNA, 21 bp, Artificial Sequence
  Description of Artificial Sequence Synthetic oligonucleotide
catgaagttctccagttccat

SEQ ID NO: 6 - DNA, 19 bp, Artificial Sequence
  Description of Artificial Sequence Synthetic oligonucleotide
agagctgctacagcatcaa

SEQ ID NO: 7 - RNA, 19 nt, Artificial Sequence
  Description of Artificial Sequence Synthetic oligonucleotide
gacauccaacagccagaaa

SEQ ID NO: 8 - RNA, 19 nt, Artificial Sequence
  Description of Artificial Sequence Synthetic oligonucleotide
gagaaagagcggccagaga

SEQ ID NO: 9 - RNA, 19 nt, Artificial Sequence
  Description of Artificial Sequence Synthetic oligonucleotide
uaccagcacuaugauugga

SEQ ID NO: 10 - RNA, 19 nt, Artificial Sequence
  Description of Artificial Sequence Synthetic oligonucleotide
ucuguauacacacggucug

SEQ ID NO: 11 - DNA, 28 bp, Artificial Sequence
  Description of Artificial Sequence Synthetic primer
ctgggccagcatggcccctgctgtggtg

SEQ ID NO: 12 - DNA, 21 bp, Artificial Sequence
  Description of Artificial Sequence Synthetic oligonucleotide
tccactgattgctgcagctaa

SEQ ID NO: 13 - DNA, 21 bp, Artificial Sequence
  Description of Artificial Sequence Synthetic oligonucleotide
ttgaagagaactgcaactgaa

SEQ ID NO: 14 - RNA, 21 nt, Artificial Sequence
  Description of Artificial Sequence Synthetic oligonucleotide
uuagcugcagcaaucagugga

SEQ ID NO: 15 - DNA, 21 bp, Artificial Sequence
  Description of combined DNA/RNA molecule Synthetic oligonucleotide
cacugauugcugcagcuaatt

SEQ ID NO: 16 - RNA, 21 nt, Artificial Sequence
  Description of Artificial Sequence Synthetic oligonucleotide
uucaguugcaguucucuucaa

SEQ ID NO: 17 - DNA, 21 bp, Artificial Sequence
  Description of combined DNA/RNA molecule Synthetic oligonucleotide
gaagagaacugcaacugaatt

SEQ ID NO: 18 - DNA, 21 bp, Artificial Sequence
  Description of Artificial Sequence Synthetic oligonucleotide
accctaaaccatggttctaaa

SEQ ID NO: 19 - DNA, 21 bp, Artificial Sequence
  Description of combined DNA/RNA molecule Synthetic oligonucleotide
uuuagaaccaugguuuagggt

SEQ ID NO: 20 - DNA, 21 bp, Artificial Sequence
  Description of combined DNA/RNA molecule Synthetic oligonucleotide
ccuaaaccaugguucuaaatt

SEQ ID NO: 21 - DNA, 21 bp, Artificial Sequence
  Description of Artificial Sequence Synthetic oligonucleotide
agccaataagcttaaagctaa

SEQ ID NO: 22 - DNA, 21 bp, Artificial Sequence
  Description of combined DNA/RNA molecule Synthetic oligonucleotide
uuagcuuuaagcuuauuggct

SEQ ID NO: 23 - DNA, 21 bp, Artificial Sequence
  Description of combined DNA/RNA molecule Synthetic oligonucleotide
ccaauaagcuuaaagcuaatt

SEQ ID NO: 24 - DNA, 21 bp, Artificial Sequence
  Description of Artificial Sequence Synthetic oligonucleotide
tcgacagtacgtggccctaaa

SEQ ID NO: 25 - RNA, 21 nt, Artificial Sequence
  Description of Artificial Sequence Synthetic oligonucleotide
uuuagggccacguacugucga

SEQ ID NO: 26 - DNA, 21 bp, Artificial Sequence
  Description of combined DNA/RNA molecule Synthetic oligonucleotide
gacaguacguggcccuaaatt

SEQ ID NO: 27 - DNA, 21 bp, Artificial Sequence
  Description of Artificial Sequence Synthetic oligonucleotide
aacgggtagttaatcctgcaa

SEQ ID NO: 28 - DNA, 21 bp, Artificial Sequence
  Description of combined DNA/RNA molecule Synthetic oligonucleotide
uugcaggauuaacuacccgtt

SEQ ID NO: 29 - DNA, 21 bp, Artificial Sequence
  Description of combined DNA/RNA molecule Synthetic oligonucleotide
cggguaguuaauccugcaatt

SEQ ID NO: 30 - DNA, 57 bp, Homo sapiens
ccgggcctggatacacatgcagtatctcgagatactgcatgtgtatccaggcttttt

SEQ ID NO: 31 - DNA, 57 bp, Homo sapiens
ccggccgagcaaatactcagatttactcgagtaaatctgagtatttgctcggttttt

SEQ ID NO: 32 - DNA, 57 bp, Homo sapiens
ccggccagggatatgaatggatattctcgagaatatccattcatatccctggttttt

SEQ ID NO: 33 - DNA, 57 bp, Homo sapiens
ccgggccacattaaagatgaagtatctcgagatacttcatctttaatgtggcttttt

SEQ ID NO: 34 - DNA, 57 bp, Homo sapiens
ccggcgtctgctagaaattgtagaactcgagttctacaatttctagcagacgttttt

SEQ ID NO: 35 - DNA, 21 bp, Artificial Sequence
  Description of Artificial Sequence Synthetic oligonucleotide
caggatgttactcaccaagaa

SEQ ID NO: 36 - DNA, 21 bp, Artificial Sequence
  Description of combined DNA/RNA molecule Synthetic oligonucleotide
uucuuggugaguaacaucctg

SEQ ID NO: 37 - DNA, 21 bp, Artificial Sequence
  Description of combined DNA/RNA molecule Synthetic oligonucleotide
ggauguuacucaccaagaatt

SEQ ID NO: 38 - DNA, 21 bp, Artificial Sequence
  Description of Artificial Sequence Synthetic oligonucleotide
ccgggtgtttcaaagacatca

SEQ ID NO: 39 - RNA, 21 nt, Artificial Sequence
  Description of Artificial Sequence Synthetic oligonucleotide
ugaugucuuugaaacacccgg

SEQ ID NO: 40 - DNA, 21 bp, Artificial Sequence
  Description of combined DNA/RNA molecule Synthetic oligonucleotide
ggguguuucaaagacaucatt

SEQ ID NO: 41 - DNA, 21 bp, Artificial Sequence
  Description of Artificial Sequence Synthetic oligonucleotide
ctcggtggtgaaagggatcta

SEQ ID NO: 42 - RNA, 21 nt, Artificial Sequence
  Description of Artificial Sequence Synthetic oligonucleotide
uagaucccuuucaccaccgag

SEQ ID NO: 43 - DNA, 21 bp, Artificial Sequence
  Description of combined DNA/RNA molecule Synthetic oligonucleotide
cgguggugaaagggaucuatt

SEQ ID NO: 44 - protein, 16 aa, Artificial Sequence
  Description of Artificial Sequence Synthetic peptide
MVDGKPVNLGLWDTAG

SEQ ID NO: 45 - DNA, 28 bp, Artificial Sequence
  Description of Artificial Sequence Synthetic primer
caccacagcaggggccatgctggcccag

SEQ ID NO: 46 - RNA, 21 nt, Artificial Sequence
  Description of Artificial Sequence Synthetic oligonucleotide
aaccugcggucuggagucaac

SEQ ID NO: 47 - RNA, 21 nt, Artificial Sequence
  Description of Artificial Sequence Synthetic oligonucleotide
ccgucaagcagaugagacauu

SEQ ID NO: 48 - RNA, 21 nt, Artificial Sequence
  Description of Artificial Sequence Synthetic oligonucleotide
ggauagagaccacgccaguuu

SEQ ID NO: 49 - RNA, 19 nt, Artificial Sequence
  Description of Artificial Sequence Synthetic oligonucleotide
gacaugauccugcaguuca